SEQUENCE LISTING (16 sequences)

SEQ ID NO: 1 - protein, 8 aa, synthetic construct
  REGION 1..8: note = Synthetic Construct
GFTFSNYA

SEQ ID NO: 2 - protein, 8 aa, synthetic construct
  REGION 1..8: note = Synthetic Construct
ISGSGDYT

SEQ ID NO: 3 - protein, 11 aa, synthetic construct
  REGION 1..11: note = Synthetic Construct
ARSPWGYYLDS

SEQ ID NO: 4 - protein, 6 aa, synthetic construct
  REGION 1..6: note = Synthetic Construct
QGISSR

SEQ ID NO: 5 - skipped

SEQ ID NO: 6 - protein, 9 aa, synthetic construct
  REGION 1..9: note = Synthetic Construct
QQYNSYPYT

SEQ ID NO: 7 - protein, 118 aa, synthetic construct
  REGION 1..118: note = Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, synthetic construct
  REGION 1..107: note = Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, synthetic construct
  REGION 1..25: note = Synthetic Construct
EVQLLESGGGLVQPGGSLRLSCAAS

SEQ ID NO: 10 - protein, 17 aa, synthetic construct
  REGION 1..17: note = Synthetic Construct
MSWVRQAPGKGLEWVSS

SEQ ID NO: 11 - protein, 38 aa, synthetic construct
  REGION 1..38: note = Synthetic Construct
YYTDSVKGRFTISRDNSKNTLYLQMNSLRAEDTAVYYC

SEQ ID NO: 12 - protein, 11 aa, synthetic construct
  REGION 1..11: note = Synthetic Construct
WGQGTLVTVSS

SEQ ID NO: 13 - protein, 26 aa, synthetic construct
  REGION 1..26: note = Synthetic Construct
DIQMTQSPPSLSASAGDRVTITCRAS

SEQ ID NO: 14 - protein, 17 aa, synthetic construct
  REGION 1..17: note = Synthetic Construct
LAWYQQKPEKAPKSLIY

SEQ ID NO: 15 - protein, 36 aa, synthetic construct
  REGION 1..36: note = Synthetic Construct
SLQSGVPSRFSGSGSGTDFTLTISSLQPEDFATYYC

SEQ ID NO: 16 - protein, 10 aa, synthetic construct
  REGION 1..10: note = Synthetic Construct
FGQGTKLEIK

(19) United States
(12) Patent Application Publication
(10) Pub. No.: US 2025/0302982 A1
(43) Pub. Date: Oct. 2, 2025

(54) METHODS OF TREATING CANCER WITH ANTI-TISSUE FACTOR ANTIBODY-DRUG CONJUGATES

(71) Applicant: Genmab A/S, Valby (DK)

(72) Inventors: Jantine BAKEMA, Utrecht (NL); Esther C.W. BREIJ, Utrecht (NL); Rieneke VAN DE VEN, Vleuten (NL); Ibrahima SOUMAORO, Plainsboro, NJ (US); Jeffrey R. HARRIS, Plainsboro, NJ (US)

(21) Appl. No.: 18/863,144

(22) PCT Filed: May 4, 2023
(86) PCT No.: PCT/EP2023/061829

§ 371 Date: Nov. 5, 2024

Related U.S. Application Data
(60) Provisional application No. 63/339,319, filed on May 6, 2022.

(51) Int. Cl. A61K 47/68; A61K 31/282; A61K 33/243; A61N 5/10; A61P 35/00

(52) U.S. Cl. CPC A61K 47/6843; A61K 31/282; A61K 33/243; A61K 47/68031; A61K 47/6889; A61N 5/10; A61P 35/00

(57) ABSTRACT

The disclosure provides antibody-drug conjugates that bind to tissue factor (TF) (e.g, tisotumab vedotin) and its use in methods of treating cancer, such as head and neck squamous cell carcinoma or a gynecological cancer, including in combination with a radiation therapy. The disclosure also provides antibody-drug conjugates that bind to TF for use in combination with an additional chemotherapeutic, such as a platinum-based agent (e.g., carboplatin or cisplatin), including in combination with a radiation therapy, for treating cancer.

### TECHNICAL FIELD

The present invention relates to methods of treating cancer with an anti-Tissue Factor (anti-TF) antibody-drug conjugate, including in combination with a radiation therapy or a chemoradiation therapy.

### BACKGROUND

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascade, and it functions as the high-affinity receptor for the coagulation factor VIIa (FVIIa), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, including head and neck squamous cell carcinoma, and is associated with more aggressive disease (see, e.g., Jacobs et al., 2012, J. Clin. Oncol. 30(15) suppl.) Furthermore, human TF also exists in a soluble alternatively-spliced form, asHTF. It has been found that asHTF promotes tumor growth (Hobbs et al., 2007, Thrombosis Res. 120(2):S13-S21).

There remains a need for improved therapies with an acceptable safety profile and high efficacy for cancer, in particular for the treatment of cancers expressing tissue factor, including gynecological cancers and head and neck cancers. The present invention meets this need by providing methods of treating cancer, such as gynecological cancers and head and neck cancers, with a combination of an anti-Tissue Factor (anti-TF) antibody-drug conjugate and radiotherapy.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject, the method comprising: (i) administering to the subject a radiation therapy; and (ii) administering to the subject an antibody-drug conjugate that binds to tissue factor (TF), wherein the antibody-drug conjugate comprises an anti-TF antibody or an antigen-binding fragment thereof conjugated to an auristatin or a functional analog thereof or a functional derivative thereof. In some embodiments, the auristatin is monomethyl auristatin or a functional analog thereof or a function derivative thereof. In some embodiments, the auristatin is monomethyl auristatin E (MMAE). In some embodiments, the antibody-drug conjugate is administered at a dose ranging from about 0.9 mg/kg to about 2.1 mg/kg. In some embodiments, the antibody-drug conjugate is administered at a dose ranging from about 0.9 mg/kg to about 1.7 mg/kg. In some embodiments, the antibody-drug conjugate is administered at a dose of about 1.3 mg/kg. In some embodiments, the antibody-drug conjugate is administered at a dose of about 1.7 mg/kg. In some embodiments, the antibody-drug conjugate is administered at a dose of about 2.0 mg/kg. In some embodiments, the antibody-drug conjugate is administered once about every 1 week, once about every 2 weeks, once about every 3 weeks or once about every 4 weeks. In some embodiments, the antibody-drug conjugate is administered once about every 2 weeks. In some embodiments, the antibody-drug conjugate is administered once about every 3 weeks. In some embodiments, the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy. In some embodiments, the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

In some embodiments of the methods, the methods further comprises administering to the subject a chemotherapeutic agent. In some embodiments, the chemotherapeutic agent is a platinum-based agent. In some embodiments, the platinum-based agent is administered at a dose between about AUC=4 and about AUC=6. In some embodiments, the platinum-based agent is administered a dose of about AUC=5. In some embodiments, the platinum-based agent is administered once about every 1 week, once about every 2 weeks, once about every 3 weeks or once about every 4 weeks. In some embodiments, the platinum-based agent is administered once about every 3 weeks. In some embodiments, the platinum-based agent is administered once about every 4 weeks.

In some embodiments of the methods, the cancer is a solid tumor. In some embodiments, the cancer is a head and neck squamous cell carcinoma. In some embodiments, the cancer is a gynecological cancer. In some embodiments, the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer. In some embodiments, the cancer is associated with a primary tumor positive for tissue factor. In some embodiments, the cancer is an early stage cancer. In some embodiments, the cancer is a stage I or stage II cancer. In some embodiments, the cancer is not a recurrent cancer. In some embodiments, the cancer is not locally advanced. In some embodiments, the cancer is not metastatic. In some embodiments, the cancer is locally advanced.

In some embodiments of the methods, the method of treating is a neoadjuvant treatment for the cancer. In some embodiments, the antibody-drug conjugate and radiation therapy are administered before surgical intervention for the cancer. In some embodiments, the platinum-based agent is further administered before surgical intervention for the cancer. In some embodiments, the antibody-drug conjugate and radiation therapy are administered before surgical removal of one or more tumors associated with the cancer. In some embodiments, the platinum-based agent is further administered before surgical removal of one or more tumors associated with the cancer. In some embodiments, the subject has not received prior therapy for the cancer.

In some embodiments of the methods, the method of treating is an adjuvant therapy for the cancer. In some embodiments, the antibody-drug conjugate and the radiation therapy are administered after surgical intervention for the cancer. In some embodiments, the platinum-based agent is further administered after surgical intervention for the cancer. In some embodiments, the antibody-drug conjugate and radiation therapy are administered after surgical removal of one or more tumors associated with the cancer. In some embodiments, the platinum-based agent is further administered after surgical removal of one or more tumors associated with the cancer.

In some embodiments of the methods, the anti-TF antibody or antigen-binding fragment thereof of the antibody-drug conjugate is a monoclonal antibody or a monoclonal antigen-binding fragment thereof. In some embodiments, the anti-TF antibody or antigen-binding fragment thereof of the antibody-drug conjugate comprises a heavy chain variable region and a light chain variable region, wherein the heavy chain variable region comprises: (i) a CDR-H1 comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and wherein the light chain variable region comprises: (i) a CDR-L1 comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6, wherein the CDRs of the anti-TF antibody or antigen-binding fragment thereof are defined by the IMGT numbering scheme. In some embodiments, the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8. In some embodiments, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO: 8. In some embodiments, the anti-TF antibody of the antibody-drug conjugate is tisotumab. In some embodiments, the antibody-drug conjugate further comprises a linker between the anti-TF antibody or antigen-binding fragment thereof and the auristatin. In some embodiments, the linker is a cleavable peptide linker. In some embodiments, the cleavable peptide linker has a formula: -MC-vc-PAB-, wherein:
a) MC is:

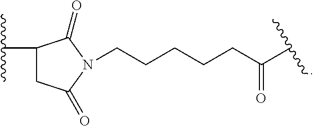 [SMILES: *C(=O)CCCCCN1C(=O)CC(*)C1=O]

b) vc is the dipeptide valine-citrulline, and
c) PAB is:

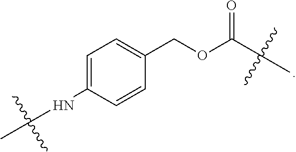 [SMILES: *Nc1ccc(COC(*)=O)cc1]

In some embodiments, the linker is attached to sulphydryl residues of the anti-TF antibody obtained by partial reduction or full reduction of the anti-TF antibody or antigen-binding fragment thereof. In some embodiments, the antibody-drug conjugate has the following structure:

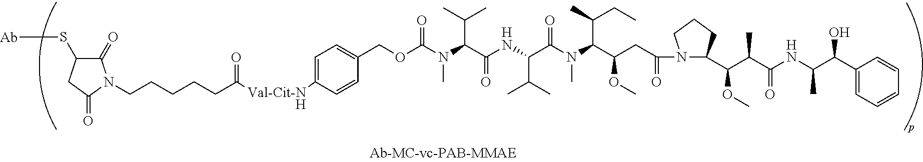

wherein p denotes a number from 1 to 8, S represents a sulphydryl residue of the anti-TF antibody, and Ab designates the anti-TF antibody or antigen-binding fragment thereof.

In some embodiments, the average value of p in a population of the antibody-drug conjugates is about 4. In some embodiments, the antibody-drug conjugate is tisotumab vedotin. In some embodiments, the route of administration for the antibody-drug conjugate is intravenous. In some embodiments, the platinum-based agent is selected from the group consisting of carboplatin, cisplatin, oxaliplatin, and nedaplatin. In some embodiments, the platinum-based agent is carboplatin. In some embodiments, the platinum-based agent is cisplatin. In some embodiments, the route of administration for the platinum-based agent is intravenous. In some embodiments, the platinum-based agent and the antibody-drug conjugate are administered sequentially. In some embodiments, the platinum-based agent and the antibody-drug conjugate are administered simultaneously. In some embodiments, the subject is a human. In some embodiments, the antibody-drug conjugate is in a pharmaceutical composition comprising the antibody-drug conjugate and a pharmaceutically acceptable carrier. In some embodiments, the platinum-based agent is in a pharmaceutical composition comprising the platinum-based agent and a pharmaceutical acceptable carrier.

### BRIEF DESCRIPTION OF THE DRAWINGS

FIG. 1A shows representative images of Tissue Factor (TF)-positive tumors (darker staining) of indicated HNSCC patients in different buckets (II-V) based on increasing TF expression. Scale bars represent 100 μm.

FIG. 1B shows heterogeneous Tissue Factor (TF) expression in tumor biopsies of HNSCC patients. A minimum of 100 tumor cells per section were manually scored for TF expression (membrane and cytoplasmic staining scored together). Percentages of TF positive cells within the tumor area were classified as I=no TF positive tumor cells (0%, white bar) or TF positive cells (black bars), II=>0-25%, III=25-50%, IV=50-75%, V=75-100%.

FIG. 2 shows the degree of tissue factor expression on the indicated HNSCC cell lines. Quantitative determination of surface expression of TF molecules on indicated HNSCC cell lines by flow cytometry using indirect immunofluorescence assay, performed in duplicate. Number of TF molecules expressed on indicated cell lines in independent experiments are indicated with filled circles. Each bar represents mean of independent experiments per cell type. * indicates HPV-positive cell lines.

FIG. 3 shows that tisotumab can bind HNSCC cells. Cell surface expression of TF on the indicated HNSCC cell lines as assessed with flow cytometry using the clinical anti-TF antibody tisotumab (black filled peaks) or isotype control IgG1, (IgG1-b12 filled grey peaks) and PE-conjugated goat (Fab′)2 anti-human IgG secondary antibody.

FIG. 4A shows dose dependent cytotoxicity of tisotumab vedotin (TV) in the indicated cell lines as assessed by percent of viable cells, and as compared to IgG1-vedotin (IgG1-V) control.

FIG. 4B shows dose dependent cytotoxicity of tisotumab vedotin (TV) in the indicated cell lines as assessed by percent of viable cells, and as compared to IgG1-vedotin (IgG1-V) control.

FIG. 4C shows the IC50 value in μg/ml of TV for each indicated cell type. Graph represents IC50 values in μg/ml of tisotumab vedotin in independent experiments. Circles indicate IC50 of separate experiments; bar represents the mean of IC50±SEM (standard error of the mean). Only those cell lines are included where a minimum of n=3 experiments was performed. * indicates HPV-positive cell lines.

FIG. 5 verifies TF expression on HNSCC cell lines (used to induce tumor growth in mice) as assessed with flow cytometry using 7.5 μg/ml human anti-TF antibody (anti-CD142-FITC; dark grey filled peaks) or stained with the isotype control-FITC (mouse IgG1-FITC) (light grey peaks).

FIG. 6 shows a schematic for a mouse tumor model experiment (“TV” is tisotumab vedotin; “RT” is radiotherapy; “CDDP” is cisplatin).

FIG. 7 shows that the tumors derived from HNSCC lines express TF. Tumors of indicated HNSCC cell line-injected mice (no treatment) were harvested and IHC analysis was performed on formalin-fixed paraffin-embedded (FFPE) tumor sections to determine TF expression using anti-CD142-FITC or isotype control (IgG1-FITC) with rabbit anti-FITC and BrightVision Immunohistochemistry (IHC) Detection Kit and visualized with 3,3′ Diaminobenzidine (DAB) counterstained with Haematoxylin. Scale bar represents 0-100 μm.

FIG. 8A shows tumor volume measured over time in different treatment groups in FaDu tumor-bearing mice. Mice were treated on day 0, 7 and 13 (black arrows) with vehicle control (PBS), indicated concentrations of tisotumab vedotin (TV) (1, 2, or 4 mg/kg), or IgG1-vedotin (IgG1-V) (4 mg/kg) control.

FIG. 8B shows mean tumor volumes (mm³)±SEM in different treatment groups in FaDu tumor-bearing mice. Mice were treated with vehicle control (PBS), indicated concentrations of tisotumab vedotin (TV) (1, 2, or 4 mg/kg), or IgG1-vedotin (IgG1-V) (4 mg/kg). Results are shown when all groups were still complete (day 7). Improved overall survival was assessed using the Kaplan-Meier method and differences between the groups as indicated by the brackets were assessed by the Log-rank test (Mantel-Cox) (*p<0.05; **p<0.01).

FIG. 8C shows tumor volume measured over time in different treatment groups in VU-SCC-OE-tumor bearing mice. Mice were treated with vehicle control (PBS), indicated concentrations of tisotumab vedotin (TV) (1, 2, or 4 mg/kg), or IgG1-vedotin (IgG1-V) (4 mg/kg). Treatment days are indicated with black arrows.

FIG. 8D shows mean tumor volumes (mm³)±SEM in different treatment groups in VU-SCC-OE-tumor bearing mice. Mice were treated with vehicle control (PBS), the indicated concentrations of tisotumab vedotin (TV) (1, 2, or 4 mg/kg), or IgG1-vedotin (IgG1-V) (4 mg/kg).

FIG. 8E shows tumor volume measured over time in different treatment groups in VU-SCC-040 tumor-bearing mice. Mice were treated on day 0, 7 and 14 (indicated by black arrows) with vehicle control (PBS), indicated concentrations of tisotumab vedotin (TV) (1, 2, or 4 mg/kg), or IgG1-vedotin IgG1-V) (4 mg/kg).

FIG. 8F shows mean tumor volumes (mm³)±SEM in different treatment groups in VU-SCC-040 tumor-bearing mice. Mice were treated with vehicle control (PBS), indicated concentrations of tisotumab vedotin (TV) (1, 2, or 4 mg/kg), or IgG1-vedotin (IgG1-V) (4 mg/kg). Results are shown when all groups were still complete (day 6).

FIG. 9A shows FaDu tumor volume measurements relative to time of complete treatment groups is indicated as a mean of tumor(s) (mm³) per mouse±SEM. FaDu bearing mice (5-6 mice/group) were treated with PBS, tisotumab vedotin (TV) (2 mg/kg, “TV2”) or IgG1-vedotin (IgG1-V) on day 0 and 10 (solid arrow). Groups labeled “CRT” received chemoradiotherapy (CRT) on day 1 and 11 (as indicated by light gray arrows).

FIG. 9B shows FaDu mean tumor volumes (mm3)±SEM in different treatment groups from FaDu-bearing mice (5-6 mice/group) which were treated with PBS, tisotumab vedotin (TV) (at 2 mg/kg, written as “TV 2”), or IgG1-vedotin (IgG1-V). Indicated “+CRT” groups received chemoradiotherapy. Kruskal-Wallis tests were performed to determine statistically significant differences between groups (*p<0.05).

FIG. 9C shows VU-SCC-OE (8 mice/group) day 9 tumor volume measurements relative to time of complete treatment groups as indicated as a mean of tumor(s) (mm³) per mouse±SEM. Mice were treated with PBS, tisotumab vedotin (TV) (at 2 mg/kg, written as “TV 2”) or IgG1-vedotin on day 0 and 10 (solid arrow). Groups labeled “+CRT” groups received chemoradiotherapy (CRT) on day 1 and 11 (light gray arrows).

FIG. 9D shows VU-SCC-OE (8 mice/group) day 14 mean tumor volumes (mm3) SEM in different treatment groups from VU-SCC-OE bearing mice which were treated with PBS, tisotumab vedotin (TV) (2 mg/kg) or IgG1-vedotin (IgG1-V) on day 0 and 10. Indicated “+CRT” groups received chemoradiotherapy on day 1 and 11. Kruskal-Wallis tests were performed to determine statistically significant differences between groups (*p<0.05; **** p<0.0001).

FIG. 9E shows FaDu tumor-bearing mouse survival plot after treatment with PBS, tisotumab vedotin (TV) (at 2 mg/kg), or IgG1-vedotin. Indicated “+CRT” groups received chemoradiotherapy (“CRT” alone indicates PBS+CRT). Survival curves were compared by log-rank (Mantel-Cox) analysis.

FIG. 9F shows a VU-SCC-OE tumor bearing mouse survival plot after treatment with PBS, tisotumab vedotin (TV) (at 2 mg/kg), or IgG1-vedotin (IgG1-V). Indicated “+CRT” groups received chemoradiotherapy. Survival curves were compared by log-rank (Mantel-Cox) analysis.

FIG. 10A shows tumor volume measurements of indicated treatment groups on day 7 after treatment with PBS, IgG1-vedotin, tisotumab vedotin (TV) (at 1 mg/kg or 2 mg/kg), with or without 2 gray (Gy) whole body radiotherapy (“RT”), or radiotherapy alone (“RT 2 Gy”). Kruskal-Wallis tests were performed to determine statistically significant differences between groups (*p<0.05; **p<0.01).

FIG. 10B shows survival curves of the different treatment groups expressed in percentages. Survival curves were compared by log-rank (Mantel-Cox) analysis. “TV2” indicated tisotumab vedotin at 2 mg/kg and “RT” indicates 2 gray (Gy) radiotherapy.

FIG. 10C shows tumor volume measurements of indicated treatment groups on day 7 after treatment. “TV1” and “TV2” indicate tisotumab vedotin dosages at 1 mg/kg and 2 mg/kg, respectively. CDDP indicates 3 mg/kg cisplatin treatment (*p<0.05; **p<0.01).

FIG. 10D shows survival curves expressed in percent survival of indicated treatment groups on day 7 after treatment. “TV1” and “TV2” indicate tisotumab vedotin dosages at 1 mg/kg and 2 mg/kg, respectively. “CDDP” or “+CT” indicates 3 mg/kg cisplatin (CDDP, chemotherapy) treatment.

### DETAILED DESCRIPTION

The present disclosure provides anti-TF antibody drug-conjugates that bind to tissue factor (TF) for use in methods of treating cancer, the method comprising administering the antibody-drug conjugate to a subject having said cancer. In some embodiments, the methods further comprise administering to the subject a radiation therapy. In some embodiments, the methods further comprise administering a radiation therapy and an additional chemotherapeutic drug, such as a platinum-based drug (e.g., cisplatin or carboplatin). In some embodiments, the cancer is a cancer positive for tissue factor. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells from the subject express TF. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cancer cells from the subject express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA). In some embodiments, the cancer is a head and neck cancer, such as head and neck squamous cellular carcinoma (HNSCC). In some embodiments, the cancer is a gynecological cancer. In some embodiments, the subject to be treated is a human.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. In some embodiments, tissue factor comprises the amino acid sequence found under Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. Unless stated otherwise, the numbering of amino acid residues in the constant region is according to the EU-index as described in Kabat et al., Sequences of Proteins of Immunological Interest, 5th Ed. Public Health Service, National Institutes of Health, Bethesda, MD. (1991). An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-TF antibody includes a conjugate of an anti-TF antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-TF antibody is an antibody that binds to the antigen TF.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-TF antibody, which is coupled to a drug moiety (e.g., MMAE or MMAF) as described in the present application.

The abbreviations “vc” and “val-cit” refer to the dipeptide valine-citrulline.

The abbreviation “PAB” refers to the self-immolative spacer:

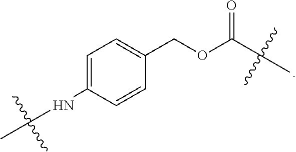 [SMILES: *Nc1ccc(COC(*)=O)cc1]

The abbreviation “MC” refers to the stretcher maleimidocaproyl:

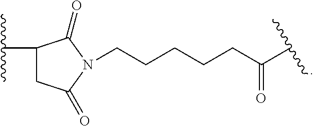 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a cervical cancer refers to a tumor that is the result of a metastasized cervical cancer.

“Treatment” or “therapy” of a subject refers to any type of intervention or process performed on, or the administration of an active agent to, the subject with the objective of curing, reversing, alleviating, ameliorating, inhibiting, slowing down, or preventing the onset, progression, development, severity, or recurrence of a symptom, complication, condition, or biochemical indicia associated with a disease. In some embodiments, the disease is cancer. A “neoadjuvant” treatment or therapy is one carried out before a main treatment (e.g., a surgical intervention) to increase the chance of favorable clinical outcomes, such as a cure, from the main treatment. In the context of cancer, for example, a neoadjuvant treatment or therapy can shrink a tumor, allowing for curative surgical intervention. An “adjuvant” treatment or therapy is one carried out after a main treatment (e.g., after a surgical intervention) in order the increase the likelihood of a cure. For example, in the context of cancer, after removal of a larger primary tumor, an adjuvant treatment or therapy may prevent the growth of secondary tumors.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns.”

A therapeutically effective amount of a drug (e.g., an anti-TF antibody-drug conjugate or a platinum-based agent) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-TF antibody-drug conjugate or a platinum-based agent) that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 2.0 mg/kg of a platinum-based agent or an anti-TF antibody-drug conjugate, one can calculate and use the appropriate amount of the platinum-based agent or anti-TF antibody-drug conjugate (i.e., 120 mg) for administration to said subject.

The use of the term “fixed dose” with regard to a method of the disclosure means that two or more different agents (e.g., a platinum-based agent and an anti-TF antibody-drug conjugate) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 3:1 ratio of a platinum-based agent to an anti-TF antibody-drug conjugate administered to a subject can mean about 240 mg of the platinum-based agent and about 80 mg of the anti-TF antibody-drug conjugate or about 3 mg/ml of the platinum-based agent and about 1 mg/ml of the anti-TF antibody-drug conjugate are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., the anti-TF antibody-drug conjugate and/or the platinum-based agent). For example, a subject with 60 kg body weight and a subject with 100 kg body weight would receive the same dose of an antibody or an antibody-drug conjugate (e.g., 240 mg of an anti-TF antibody-drug conjugate or e.g. 750 mg of a platinum-based agent).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-TF antibody-drug conjugate and/or platinum-based agent include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-TF antibody-drug conjugate as described herein and/or a platinum-based agent as described herein) or at the beginning of administration of the therapy. The baseline value can be compared to a reference value in order to determine the reduction or improvement of a symptom of a disease contemplated herein, such as TF-associated disease contemplated herein (e.g., a cancer). The terms “reference” or “reference value” used interchangeably herein can refer to a measurement or characterization of a symptom after administration of the therapy (e.g., an anti-TF antibody-drug conjugate as described herein and/or a platinum-based agent as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-TF antibody-drug conjugate or platinum-based agent is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Antibody-Drug Conjugates

The present disclosure provides anti-TF antibody-drug conjugates that bind to TF for use in the treatment of cancer, wherein the treatment comprises administering to a subject having said cancer the antibody-drug conjugate, and, in some embodiments, wherein the method further comprises administering to the subject a radiation therapy. In some embodiments, the addition of the antibody-drug conjugate to the treatment comprising a radiation therapy enhances the efficacy of the radiation therapy. In some embodiments, the antibody-drug conjugate comprises an anti-TF antibody or an antigen-binding fragment thereof conjugated to an auristatin (such as monomethyl auristatin) or a functional analog thereof or a functional derivative thereof. In some embodiments, the cancer is associated with a tumor that is positive for tissue factor. In some embodiments, the cancer is a head and neck cancer. In some embodiments, the head and neck cancer is head and neck squamous cellular carcinoma (HNSCC). In some embodiments, the cancer is a gynecological cancer. In some embodiments, the gynecological cancer is selected from ovarian cancer, endometrial cancer, cervical cancer, perineal tissue cancer, fallopian tube cancer, uterine cancer, vaginal cancer, vulvar cancer, and gestational trophoblastic disease cancer. In some embodiments, the gynecological cancer is an ovarian cancer. In some embodiments, the gynecological cancer is an endometrial cancer. In some embodiments, the gynecological cancer is a cervical cancer. In some embodiments, the gynecological cancer is a perineal tissue cancer. In some embodiments, the gynecological cancer is a fallopian tube cancer. In some embodiments, the cancer is a uterine cancer. In some embodiments, the gynecological cancer is a vaginal cancer. In some embodiments, the gynecological cancer is a vulvar cancer. In some embodiments, the gynecological cancer is a gestational trophoblastic disease cancer. In some embodiments, the cancer is an early stage cancer (such as stage I or stage II). In some embodiments, the cancer is not recurrent. In some embodiments, the cancer is locally advanced. In some embodiments, the cancer is not metastatic. In some embodiments, the treatment is a neoadjuvant treatment (such as a therapy prior to a surgical intervention). In some embodiments the treatment is an adjuvant therapy (such as following a surgical intervention). In some embodiments, the radiation therapy is selected from the group consisting of intensity-modulated radiation therapy (IMRT), image-guided radiation therapy (IGRT), tomotherapy, stereotactic radiosurgery, stereotactic body radiation therapy, photon beam therapy, electron beam therapy, and proton therapy. In some embodiments, the radiation therapy is intensity-modulated radiation therapy (IMRT). In some embodiments, the radiation therapy is image-guided radiation therapy (IGRT). In some embodiments, the radiation therapy is tomotherapy. In some embodiments, the radiation therapy is stereotactic radiosurgery. In some embodiments, the radiation therapy is stereotatic body radiation therapy. In some embodiments, the radiation therapy is photon beam therapy. In some embodiments, the radiation therapy is electron beam therapy. In some embodiments, the radiation therapy is proton therapy.

In some embodiments, the antibody-drug conjugate of the present disclosure is to be administered to patients who have received, are receiving, or will receive a radiation therapy. In some embodiments, in external beam radiotherapy, high energy rays (e.g., gamma-rays or X-rays) and/or energetic/charged particles may be generated outside the subject and delivered to a target tumor associated with the cancer being treated. Tumor cells, and healthy cells, within the path of the beam will absorb some of the radiation. In some embodiments, to avoid damage to surrounding healthy tissue (collateral tissue damage), the target tumor area may be treated with lower levels of radiation administered from different points of entrance (vectors of radiation). In some embodiments, the radiation dose may be “fractionated” by delivering a portion of the dose in one fractional dose, and delivering the remainder of the dose in one or more additional fractional doses, typically from different approach vector(s) or at different times (to give healthy tissue time to recover). In some embodiments, stereotactic body radiation therapy is used to apply image-guided, focused high-dose external beam X-ray radiation to target tumors in a small area, often in a single fraction. In some embodiments, stereotactic radiosurgery is used as a non-surgical approach that delivers a single high-dose of radiation, typically to the brain, head, and/or neck using highly focused gamma-ray or X-ray beams that converge on the specific site where the tumor resides. In some embodiments, intraoperative radiation therapy is used as an approach delivering radiation using a focused high-dose radiation using a beam of ionizing radiation directed to the tumor site while the site is exposed during surgery.

In some embodiments, the radiation used in the radiotherapy may be selected from any type suitable for treating cancer. In some embodiments, radiation may be delivered from a machine outside the body (external radiation) or from a machine placed in the body (internal radiation). In some embodiments, the type of radiation used will depend upon the position in the body, the amount of healthy tissue to be penetrated, the particular cancer, and the particular subject. In some embodiments, multiple types of radiation may be used as part of the radiotherapy.

The dose of radiation delivered in the radiotherapy may range from about 1 Gy to about 100 Gy, and any values and ranges therebetween. In some embodiments, the dose of radiation delivered is from about 1 Gy to about 100 Gy, including about 1 Gy to about 60 Gy, about 1 Gy to about 50 Gy, about 1 Gy to about 40 Gy, about 1 Gy to about 30 Gy, about 1 Gy to about 20 Gy, about 1 Gy to about 10 Gy, about 10 Gy to about 90 Gy, about 10 Gy to about 80 Gy, about 10 Gy to about 70 Gy, about 10 Gy to about 60 Gy, about 10 Gy to about 50 Gy, about 10 Gy to about 40 Gy, about 10 Gy to about 30 Gy, about 20 Gy to about 90 Gy, about 20 Gy to about 80 Gy, about 20 Gy to about 70 Gy, about 20 Gy to about 60 Gy, about 20 Gy to about 50 Gy, about 20 Gy to about 40 Gy, about 30 Gy to about 90 Gy, about 30 Gy to about 80 Gy, about 30 Gy to about 70 Gy, about 30 Gy to about 60 Gy, about 40 Gy to about 90 Gy, about 40 Gy to about 80 Gy, about 40 Gy to about 70 Gy, about 40 Gy to about 60 Gy, about 40 Gy to about 50 Gy, about 50 Gy to about 90 Gy, about 50 Gy to about 80 Gy, or about 50 Gy to about 70 Gy, and any values and ranges there between. In some embodiments, the dose of radiation delivered is less than about 100 Gy, such as less than about 90 Gy, less than about 80 Gy, less than about 70 Gy, less than about 60 Gy, less than about 50 Gy, less than about 40 Gy, less than about 30 Gy, less than about 20 Gy, and less than about 10 Gy. In some embodiments, the dose of radiation delivered is less than about 90 Gy. In some embodiments, the dose of radiation delivered is less than about 80 Gy. In some embodiments, the dose of radiation delivered is less than about 70 Gy. In some embodiments, the dose of radiation delivered is less than about 60 Gy. In some embodiments, the dose of radiation delivered is less than about 50 Gy. In some embodiments, the dose of radiation delivered is less than about 40 Gy. In some embodiments, the dose of radiation delivered is less than about 30 Gy. In some embodiments, the dose of radiation delivered is less than about 20 Gy. In some embodiments, the dose of radiation delivered is less than about 10 Gy. In some embodiments, the dose of radiation delivered is about 5 Gy. In some embodiments, the dose of radiation delivered is about 10 Gy. In some embodiments, the dose of radiation delivered is about 15 Gy. In some embodiments, the dose of radiation delivered is about 20 Gy. In some embodiments, the dose of radiation delivered is about 25 Gy. In some embodiments, the dose of radiation delivered is about 30 Gy. In some embodiments, the dose of radiation delivered is about 35 Gy. In some embodiments, the dose of radiation delivered is about 40 Gy. In some embodiments, the dose of radiation delivered is about 45 Gy. In some embodiments, the dose of radiation delivered is about 50 Gy. In some embodiments, the dose of radiation delivered is about 55 Gy. In some embodiments, the dose of radiation delivered is about 60 Gy. In some embodiments, the dose of radiation delivered is about 65 Gy. In some embodiments, the dose of radiation delivered is about 70 Gy. In some embodiments, the dose of radiation delivered is about 75 Gy. In some embodiments, the dose of radiation delivered is about 80 Gy. In some embodiments, the dose of radiation delivered is about 85 Gy. In some embodiments, the dose of radiation delivered is about 90 Gy. In some embodiments, the dose of radiation delivered is about 95 Gy. In some embodiments, the dose of radiation delivered is about 100 Gy. In some embodiments, the dose of radiation delivered is 5 Gy. In some embodiments, the dose of radiation delivered is 10 Gy. In some embodiments, the dose of radiation delivered is 15 Gy. In some embodiments, the dose of radiation delivered is 20 Gy. In some embodiments, the dose of radiation delivered is 25 Gy. In some embodiments, the dose of radiation delivered is 30 Gy. In some embodiments, the dose of radiation delivered is 35 Gy. In some embodiments, the dose of radiation delivered is 40 Gy. In some embodiments, the dose of radiation delivered is 45 Gy. In some embodiments, the dose of radiation delivered is 50 Gy. In some embodiments, the dose of radiation delivered is 55 Gy. In some embodiments, the dose of radiation delivered is 60 Gy. In some embodiments, the dose of radiation delivered is 65 Gy. In some embodiments, the dose of radiation delivered is 70 Gy. In some embodiments, the dose of radiation delivered is 75 Gy. In some embodiments, the dose of radiation delivered is 80 Gy. In some embodiments, the dose of radiation delivered is 85 Gy. In some embodiments, the dose of radiation delivered is 90 Gy. In some embodiments, the dose of radiation delivered is 95 Gy. In some embodiments, the dose of radiation delivered is 100 Gy. In some embodiments, the radiation dose delivered is the sum of any fractional doses delivered as part of the dose. In some embodiments, the radiation dose may be reduced by delivering a radiosensitizer to the target tumor or target tumor site.

In some embodiments, the radiation dose may be administered in 1 to about 60 fractional doses. In some embodiments, the radiation dose is delivered in 1 dose. In some embodiments, the radiation dose is fractionated and delivered from about 2 to about 10 fractional doses. In some embodiments, the radiation dose is fractionated and delivered from about 5 to about 60 fractional doses. In some embodiments, the radiation dose is fractionated and delivered in fractional doses of about 1 Gy to about 20 Gy. In some embodiments, the interval between fractional doses may be on the order of minutes, hours, days, or weeks. In some embodiments, the fractional doses are administered with intervals less than 10 minutes. In some embodiments, the fractional doses are administered with intervals from about 1 hour to about 1 week. In some embodiments, the fractional doses are administered with intervals from about 1 week to about 26 weeks.

In some embodiments, the source of the radiation may be selected from gamma-rays and X-rays. X-rays may be created by linear accelerators. In some embodiments, gamma-rays may be created by selecting radioactive isotopes, such as cobalt 60. In some embodiments, the source of the radiation is gamma-rays. In some embodiments, the source of the radiation is X-rays. In some embodiments, the source of the radiation may be particle beams. Particle beams may be created by linear accelerators, synchrotrons, betatrons, cyclotrons, and the like, each of which accelerate particles for delivery to the subject. In some embodiments, particle beams may be created by linear accelerators. In some embodiments, particle beams may be created by synchrotrons. In some embodiments, particle beams may be created by betatrons. In some embodiments, particle beams may be created by cyclotrons. In some embodiments, particle beams may be generated by electrons or neutrons.

In some embodiments, the radiation therapy comprises a stereotactic radiosurgery. In stereotactic radiosurgery, the subject's head can be placed in a device which is used to aim high-dose radiation beams directly at the tumor in the subject's head. In some embodiments, an imaging system may be used in conjunction with the movement of an accelerator to precisely deliver radiation to the target tumor site. In some embodiments, the radiation therapy may be stereotactic body radiation therapy, which uses a similar approach to stereotactic radiosurgery except that multiple small doses fractional doses of radiation, as opposed to one large dose, are typically used and delivered to potential target sites in the body.

In some embodiments, the radiation therapy comprises an intensity-modulated radiation therapy (IMRT). IMRT uses radiation beams, such as X-ray beams, of varying intensities to deliver different doses of radiation to small areas of tissue at the same time. This approach allows for higher doses to be delivered to the tumor and lower doses to be delivered to surrounding healthy tissue.

### III. Anti-TF Antibody

Generally, anti-TF antibodies of the disclosure bind Tissue Factor (TF, e.g., human TF, CD142), and exert cytostatic and cytotoxic effects on malignant cells, such as head and neck or gynecological cancer cells. Anti-TF antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. In some embodiments, the anti-TF antibodies of the disclosure specifically bind TF. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-TF antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-TF antibodies or antigen-binding fragments thereof are human, murine (e.g., mouse and rat), donkey, sheep, rabbit, goat, guinea pig, camelid, horse, or chicken.

The anti-TF antibodies of the present disclosure may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-TF antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

Numbering of amino acid residues in CDR sequences provided herein are according to the IMGT numbering scheme as described in Lefranc, M. P. et al., Dev. Comp. Immunol., 2003, 27, 55-77. CDR sequences provided herein for the anti-TF antibodies of the anti-TF antibody-drug conjugate are according to the IMGT method as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof binds to TF. In some embodiments, said antibody or derivative thereof specifically binds to TF. In certain embodiments, the anti-TF antibody is 011. The antibody 011 is also known as tisotumab.

In one aspect, anti-TF antibodies that compete with tisotumab binding to TF are also provided herein. Anti-TF antibodies that bind to the same epitope as tisotumab are also provided herein.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6, wherein the CDRs of the anti-TF antibody are defined by the IMGT numbering scheme.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO:13, 14, 15, and 16 (LC-FR1, LC-FR2, LC-FR3, and LC-FR4, respectively).

In some embodiments of the anti-TF antibodies described herein, the heavy chain variable domain comprises the amino acid sequence of EVQLLESGGGLVQPGGSLRLSCAASGFTFSNYAMSWVRQAPGKGLEWVSSISGSGD YTYYTDSVKGRFTISRDNSKNTLYLQMNSLRAEDTAVYYCARSPWGYYLDSWGQG TLVTVSS (SEQ ID NO:7) and the light chain variable domain comprises the amino acid sequence of DIQMTQSPPSLSASAGDRVTITCRASQGISSRLAWYQQKPEKAPKSLIYAASSLQSGV PSRFSGSGSGTDFTLTISSLQPEDFATYYCQQYNSYPYTFGQGTKLEIK (SEQ ID NO:8).

In some embodiments of the anti-TF antibodies described herein, the heavy chain CDR sequences comprise the following:

a) CDR-H1
(GFTFSNYA (SEQ ID NO: 1));
b) CDR-H2
(ISGSGDYT (SEQ ID NO: 2));
and
c) CDR-H3
(ARSPWGYYLDS (SEQ ID NO: 3)).

In some embodiments of the anti-TF antibodies described herein, the heavy chain FR sequences comprise the following:

a) HC-FR1
(EVQLLESGGGLVQPGGSLRLSCAAS (SEQ ID NO: 9));
b) HC-FR2
(MSWVRQAPGKGLEWVSS (SEQ ID NO: 10));
c) HC-FR3
(YYTDSVKGRFTISRDNSKNTLYLQMNSLRAEDTAVYYC; (SEQ
ID NO: 11));
and
d) HC-FR4
(WGQGTLVTVSS (SEQ ID NO: 12)).

In some embodiments of the anti-TF antibodies described herein, the light chain CDR sequences comprise the following:

a) CDR-L1
(QGISSR (SEQ ID NO: 4));
b) CDR-L2
(AAS (SEQ ID NO: 5));
and
c) CDR-L3
(QQYNSYPYT (SEQ ID NO: 6)).

In some embodiments of the anti-TF antibodies described herein, the light chain FR sequences comprise the following:

a) LC-FR1
(DIQMTQSPPSLSASAGDRVTITCRAS (SEQ ID NO: 13);
b) LC-FR2
(LAWYQQKPEKAPKSLIY (SEQ ID NO: 14));
c) LC-FR3
(SLQSGVPSRFSGSGSGTDFTLTISSLQPEDFATYYC (SEQ ID
NO: 15));
and
d) LC-FR4
(FGQGTKLEIK (SEQ ID NO: 16)).

In some embodiments, provided herein is an anti-TF antibody that binds to TF (e.g., human TF), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO: 9;
   (2) an CDR-H1 comprising the amino acid sequence of SEQ ID NO: 1;
   (3) an HC-FR2 comprising the amino acid sequence of SEQ ID NO:10;
   (4) an CDR-H2 comprising the amino acid sequence of SEQ ID NO: 2;
   (5) an HC-FR3 comprising the amino acid sequence of SEQ ID NO: 11;
   (6) an CDR-H3 comprising the amino acid sequence of SEQ ID NO:3; and
   (7) an HC-FR4 comprising the amino acid sequence of SEQ ID NO: 12,
and/or
(b) alight chain variable domain comprising:
   (1) an LC-FR1 comprising the amino acid sequence of SEQ ID NO: 13;
   (2) an CDR-L1 comprising the amino acid sequence of SEQ ID NO: 4;
   (3) an LC-FR2 comprising the amino acid sequence of SEQ ID NO: 14;
   (4) an CDR-L2 comprising the amino acid sequence of SEQ ID NO: 5;
   (5) an LC-FR3 comprising the amino acid sequence of SEQ ID NO: 15;
   (6) an CDR-L3 comprising the amino acid sequence of SEQ ID NO: 6; and
   (7) an LC-FR4 comprising the amino acid sequence of SEQ ID NO: 16.

In one aspect, the anti-TF antibody comprises a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDRs (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 comprising the amino acid sequence of SEQ ID NO: 1, a heavy chain CDR2 comprising the amino acid sequence of SEQ ID NO: 2, a heavy chain CDR3 comprising the amino acid sequence of SEQ ID NO: 3; and ii) a light chain CDR1 comprising the amino acid sequence of SEQ ID NO: 4, a light chain CDR2 comprising the amino acid sequence of SEQ ID NO: 5, and a light chain CDR3 comprising the amino acid sequence of SEQ ID NO: 6, wherein the CDRs of the anti-TF antibody are defined by the IMGT numbering scheme.

In some embodiments, the anti-TF antibody of the anti-TF antibody-drug conjugate comprises: i) an amino acid sequence having at least 85% sequence identity to a heavy chain variable region comprising the amino acid sequence of SEQ ID NO: 7, and ii) an amino acid sequence having at least 85% sequence identity to a light chain variable region comprising the amino acid sequence of SEQ ID NO: 8.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Anti-TF antibodies of the present invention may also be described or specified in terms of their binding affinity to TF (e.g., human TF, CD142). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### IV. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:
a) MC is:

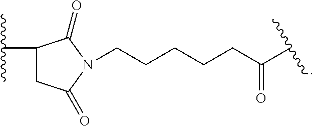 [SMILES: *C(=O)CCCCCN1C(=O)CC(*)C1=O]

b) vc is the dipeptide valine-citrulline, and
c) PAB is:

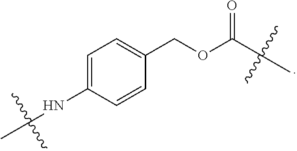 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:
a) MC is:

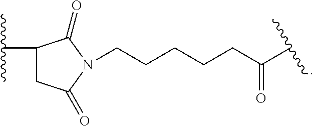 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. No. 5,663,149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

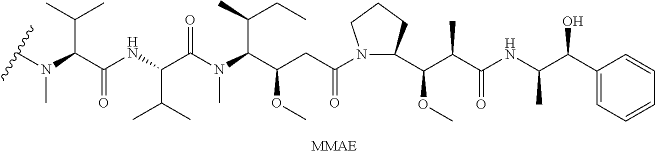 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

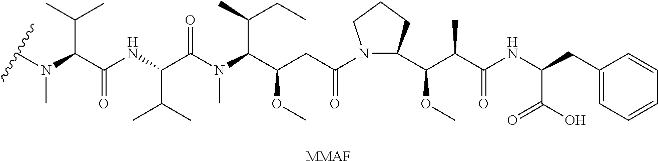

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

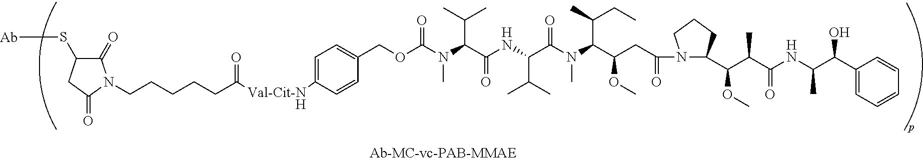

wherein p denotes a number from 1 to 8, e.g., 1, 2, 3, 4, 5, 6, 7 or 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. In another embodiment, a non-cleavable linker MC is attached to MMAF. The resulting linker-auristatin MC-MMAF is also designated mcMMAF. Both the vcMMAF and mcMMAF drug linker moieties and conjugation methods are disclosed in WO2005081711 and U.S. Pat. No. 7,498,298. When vcMMAF or mcMMAF is attached to an anti-TF antibody or antigen-binding fragment thereof as described herein, the resulting structure is:

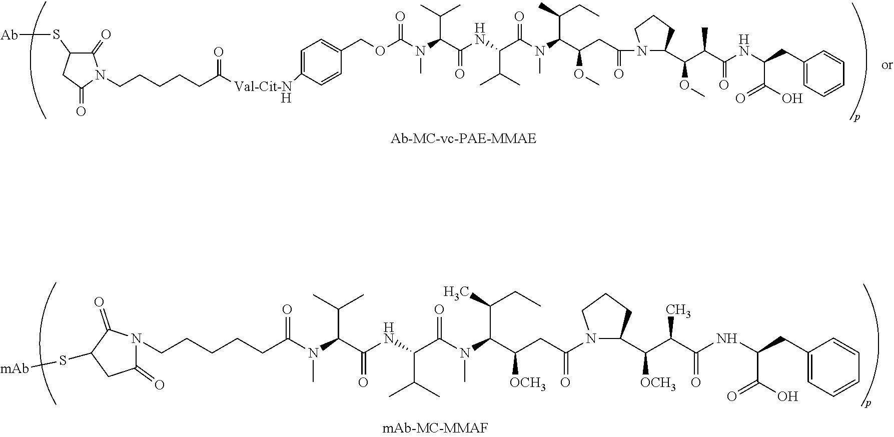

wherein p denotes a number from 1 to 8, e.g., 1, 2, 3, 4, 5, 6, 7 or 8, e.g., p may be from 3-5, S represents a sulphydryl residue of the anti-TF antibody and Ab or mAb designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

Methods of producing an anti-TF antibody, linker and anti-TF antibody-drug conjugate are described in U.S. Pat. No. 9,168,314.

The anti-TF antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-TF antibodies from the cell media using well known techniques in the art, they are conjugated with an auristatin via a linker as described in U.S. Pat. No. 9,168,314.

Monoclonal anti-TF antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J Mol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-TF antibody) of the invention is a human antibody. Human monoclonal antibodies directed against TF may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG,κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain transgenes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KCO5) 9272 trans gene (Fishwild et al. (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KC005 [MIK](Balb) mice which were generated by backcrossing the KC05 strain (as described in Fishwild et al. (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### V. Additional Chemotherapeutics

In addition to administering the antibody-drug conjugate and a radiation therapy, the methods of the disclosure may, in some embodiments, further comprise administering an additional chemotherapeutic agent. In some embodiments, the additional chemotherapeutic agent is selected from the group consisting of pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some preferred embodiments, the additional chemotherapeutic agent is a platinum-based agent. Generally, a platinum-based agent of the disclosure is a molecule or a composition comprising a molecule containing a coordination complex comprising the chemical element platinum and useful as a chemotherapy drug. In some embodiments, the platinum-based agent binds covalently to DNA and cross-links strands, inhibits DNA synthesis, and/or inhibits transcript. Platinum-based agents encompass those that are currently being used as part of a chemotherapy regimen, those that are currently in development, and those that may be developed in the future. Platinum-based agents include, but are not limited to, carboplatin, cisplatin, oxaliplatin, nedaplatin, triplatin tetranitrate, phenanthriplatin, picoplatin and satraplatin. In some preferred embodiments, the platinum-based agent is carboplatin, cisplatin, oxaliplatin or nedaplatin. In particularly preferred embodiments, the platinum-based agent is carboplatin. In other particularly preferred embodiments, the platinum-based agent is cisplatin. In some embodiments, the platinum-based agent is oxaliplatin. In some embodiments, the platinum-based agent is nedaplatin.

### VI. Routes of Administration

The anti-TF antibody-drug conjugate or antigen-binding fragment thereof described herein and the additional chemotherapeutic agent, if used, can be administered by any suitable route and mode. Suitable routes of administering are well known in the art and may be selected by those of ordinary skill in the art. In one embodiment, anti-TF antibody-drug conjugate and/or additional chemotherapeutic agent are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-TF antibody-drug conjugate or antigen-binding fragment described herein is intravenous injection or infusion. In some embodiments, the route of administration of an anti-TF antibody-drug conjugate or antigen-binding fragment described herein is intravenous infusion. In some embodiments, the route of administration of a platinum-based agent described herein is intravenous injection or infusion. In some embodiments, the route of administration of an additional chemotherapeutic agent described herein is intravenous infusion.

### VII. Dosage and Frequency of Administration

The present disclosure provides for methods of treating a subject with cancer as described herein with a particular dose of an anti-TF antibody-drug conjugate or antigen-binding fragment thereof and a radiation therapy as described herein, wherein the subject is administered the antibody-drug conjugate or antigen-binding fragment thereof as described herein, and optionally an additional chemotherapeutic agent, with particular frequencies.

In one embodiment of the methods or uses or product for uses provided herein, an anti-TF antibody-drug conjugate or antigen-binding fragment thereof as described herein is administered to the subject at a dose ranging from about 0.9 mg/kg to about 2.1 mg/kg of the subject's body weight. In certain embodiments, the dose is about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 2.0 mg/kg. In certain embodiments, the dose is 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is 2.0 mg/kg. In some preferred embodiments, the dose is 2.0 mg/kg and the anti-TF antibody-drug conjugate is tisotumab vedotin. In one embodiment, the dose is about 1.3 mg/kg. In other preferred embodiments, the dose is 1.3 mg/kg. In some embodiments, the dose is 1.3 mg/kg and the anti-TF antibody-drug conjugate is tisotumab vedotin. In some other preferred embodiments, the dose is about 1.7 mg/kg. In some embodiments, the dose is 1.7 mg/kg. In some embodiments, the dose is 1.7 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses or product for uses provided herein, an anti-TF antibody-drug conjugate or antigen-binding fragment thereof as described herein is administered to the subject once about every 1 to 4 weeks. In some embodiments, the therapy is an adjuvant therapy following surgery and may be in combination with radiation therapy. In some embodiments, the therapy is a neoadjuvant therapy preceding surgery and may be in combination with radiation therapy. In some embodiments, the therapy may be in combination with a platinum-based drug, such as carboplatin or cisplatin. In certain embodiments, an anti-TF antibody-drug conjugate or antigen-binding fragment thereof as described herein is administered once about every 1 week, once about every 2 weeks, once about every 3 weeks or once about every 4 weeks. In one embodiment, an anti-TF antibody-drug conjugate or antigen-binding fragment thereof as described herein is administered once about every 3 weeks. In one embodiment, an anti-TF antibody-drug conjugate or antigen-binding fragment thereof as described herein is administered once every 3 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., ±3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 1.3 mg/kg and is administered once every 3 weeks. In some embodiments, the dose is 1.3 mg/kg and is administered once every 3 weeks and the antibody-drug conjugate is tisotumab vedotin. In some embodiments, the dose is 1.3 mg/kg and is administered once every 3 weeks and the antibody-drug conjugate is tisotumab vedotin and the dose is decreased to 0.9 mg/kg if one or more adverse events occur. In some embodiments, the dose is 1.7 mg/kg and is administered once every 2 weeks. In some embodiments, the dose is 1.7 mg/kg and is administered once every 2 weeks and the antibody-drug conjugate is tisotumab vedotin. In some embodiments, the dose is 1.7 mg/kg and is administered once every 2 weeks and the antibody-drug conjugate is tisotumab vedotin and the dose is decreased to 1.3 mg/kg if one or more adverse events occur. In some embodiments, the dose is 1.7 mg/kg and is administered once every 2 weeks. In some embodiments, the dose is 1.7 mg/kg and is administered once every 2 weeks and the antibody-drug conjugate is tisotumab vedotin. In some embodiments, such dosing of the antibody-drug conjugate described above is provided as a neoadjuvant therapy prior to a surgical intervention. In some embodiments, such dosing of the antibody-drug conjugate described above is provided as an adjuvant therapy after a surgical intervention. In some embodiments, such dosing of the antibody-drug conjugate described above is in combination with a radiation therapy and is provided as a neoadjuvant therapy prior to a surgical intervention. In some embodiments, such dosing of the antibody-drug conjugate described above is in combination with a radiation therapy and is provided as an adjuvant therapy after a surgical intervention. In some embodiments, such dosing of the antibody-drug conjugate described above is in combination with a radiation therapy and a platinum-based drug (such as cisplatin or carboplatin) and is provided as a neoadjuvant therapy prior to a surgical intervention. In some embodiments, such dosing of the antibody-drug conjugate described above is in combination with a radiation therapy and a platinum-based drug (such as cisplatin or carboplatin) and is provided as an adjuvant therapy after a surgical intervention.

In one embodiment of the methods or uses or product for uses provided herein, an anti-TF antibody-drug conjugate or antigen-binding fragment thereof as described herein is administered to the subject at a flat dose ranging from about 50 mg to about 200 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 110 mg or a flat dose of about 120 mg or a flat dose of about 130 mg or a flat dose of about 140 mg or a flat dose of about 150 mg or a flat dose of about 160 mg or a flat dose of about 170 mg or a flat dose of about 180 mg or a flat dose of about 190 mg or a flat dose of about 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., ±3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses or product for uses provided herein, an anti-TF antibody-drug conjugate or antigen-binding fragment thereof as described herein is administered to the subject at a flat dose ranging from 50 mg to 200 mg such as at a flat dose of 50 mg or a flat dose of 60 mg or a flat dose of 70 mg or a flat dose of 80 mg or a flat dose of 90 mg or a flat dose of 100 mg or a flat dose of 110 mg or a flat dose of 120 mg or a flat dose of 130 mg or a flat dose of 140 mg or a flat dose of 150 mg or a flat dose of 160 mg or a flat dose of 170 mg or a flat dose of 180 mg or a flat dose of 190 mg or a flat dose of 200 mg. In some embodiments, the flat dose is administered to the subject once about every 1 to 4 weeks. In certain embodiments, the flat dose is administered to the subject once about every 1 week, once about every 2 weeks, once about every 3 weeks or once about every 4 weeks. In some embodiments, the flat dose is administered to the subject once about every 3 weeks (e.g., 3 days). In some embodiments, the flat dose is administered to the subject once every 3 weeks. In some embodiments, the flat dose is administered to the subject once every 3 weeks and the antibody-drug conjugate is tisotumab vedotin.

In one embodiment of the methods or uses or product for uses provided herein, a platinum-based agent described herein, such as carboplatin, is administered to the subject at a dose based on the Calvert formula:

$$Platinum - based agent dose ( mg ) = ( Target AUC ) \times ( GFR + 25 )$$

wherein AUC stands for “area under the concentration versus time curve” (AUC is expressed in mg/mL·min) and GFR stands for “glomular filtration rate” (GFR is expressed in mL/min). In some embodiments, GFR is estimated by calculated creatine clearance. In some embodiments, serum creatine is measured by the IDMS method. In some embodiments, the platinum-based agent described herein, such as carboplatin, is administered (in addition to administering the antibody-drug conjugate described herein or the antibody-drug conjugate and the radiation therapy described herein) at a dose between about AUC=4 and about AUC=6. In some embodiments, the dose of the platinum-based agent described herein, such as carboplatin, is about any of AUC=4, AUC=4.5, AUC=5, AUC=5.5, or AUC=6. In some embodiments, the dose of the platinum-based agent described herein, such as carboplatin, is about AUC=5. In some embodiments, the dose of the platinum-based agent described herein, such as carboplatin, is AUC=5. In some embodiments, the dose is about AUC=4 and is administered once about every 1 week. In some embodiments, the dose is about AUC=4 and is administered once about every 2 week. In some embodiments, the dose is about AUC=4 and is administered once about every 3 week. In some embodiments, the dose is about AUC=4 and is administered once about every 4 week. In some embodiments, the dose is about AUC=4.5 and is administered once about every 1 week. In some embodiments, the dose is about AUC=4.5 and is administered once about every 2 week. In some embodiments, the dose is about AUC=4.5 and is administered once about every 3 week. In some embodiments, the dose is about AUC=4.5 and is administered once about every 4 week. In some embodiments, the dose is about AUC=5 and is administered once about every 1 week. In some embodiments, the dose is about AUC=5 and is administered once about every 2 week. In some embodiments, the dose is about AUC=5 and is administered once about every 3 week. In some embodiments, the dose is about AUC=5 and is administered once about every 4 week. In some embodiments, the dose is about AUC=5.5 and is administered once about every 1 week. In some embodiments, the dose is about AUC=5.5 and is administered once about every 2 week. In some embodiments, the dose is about AUC=5.5 and is administered once about every 3 week. In some embodiments, the dose is about AUC=5.5 and is administered once about every 4 week. In some embodiments, the dose is about AUC=6 and is administered once about every 1 week. In some embodiments, the dose is about AUC=6 and is administered once about every 2 week. In some embodiments, the dose is about AUC=6 and is administered once about every 3 week. In some embodiments, the dose is about AUC=6 and is administered once about every 4 week. In some embodiments, the dose of the platinum-based agent described herein, such as carboplatin, is any of AUC=4, AUC=4.5, AUC=5, AUC=5.5, or AUC=6. In some embodiments, the dose of the platinum-based agent described herein, such as carboplatin, is AUC=5. In some embodiments, the dose of the platinum-based agent described herein, such as carboplatin, is AUC=5. In some embodiments, the dose is AUC=4 and is administered once about every 1 week. In some embodiments, the dose is AUC=4 and is administered once about every 2 week. In some embodiments, the dose is AUC=4 and is administered once about every 3 week. In some embodiments, the dose is AUC=4 and is administered once about every 4 week. In some embodiments, the dose is AUC=4.5 and is administered once about every 1 week. In some embodiments, the dose is AUC=4.5 and is administered once about every 2 week. In some embodiments, the dose is AUC=4.5 and is administered once about every 3 week. In some embodiments, the dose is AUC=4.5 and is administered once about every 4 week. In some embodiments, the dose is AUC=5 and is administered once about every 1 week. In some embodiments, the dose is AUC=5 and is administered once about every 2 week. In some embodiments, the dose is AUC=5 and is administered once about every 3 week. In some embodiments, the dose is AUC=5 and is administered once about every 4 week. In some embodiments, the dose is AUC=5.5 and is administered once about every 1 week. In some embodiments, the dose is AUC=5.5 and is administered once about every 2 week. In some embodiments, the dose is AUC=5.5 and is administered once about every 3 week. In some embodiments, the dose is AUC=5.5 and is administered once about every 4 week. In some embodiments, the dose is AUC=6 and is administered once about every 1 week. In some embodiments, the dose is AUC=6 and is administered once about every 2 week. In some embodiments, the dose is AUC=6 and is administered once about every 3 week. In some embodiments, the dose is AUC=6 and is administered once about every 4 week. In some embodiments, the dose is AUC=5 and is administered once about every 3 weeks (e.g., 3 days). In some embodiments, the dose is AUC=5 and is administered once every 3 weeks. In some embodiments, the dose is AUC=5 and is administered once every 3 weeks and the platinum-based agent is carboplatin.

In one embodiment of the methods or uses or product for uses provided herein, a platinum-based agent as described herein is administered to the subject at flat dose ranging from about 50 mg to about 900 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 120 mg or a flat dose of about 140 mg or a flat dose of about 160 mg or a flat dose of about 180 mg or a flat dose of about 200 mg or a flat dose of about 220 mg or a flat dose of about 240 mg or a flat dose of about 260 mg or a flat dose of about 280 mg or a flat dose of about 300 mg or a flat dose of about 320 mg or a flat dose of about 340 mg or a flat dose of about 360 mg or a flat dose of about 380 mg or a flat dose of about 400 mg or a flat dose of about 420 mg or a flat dose of about 440 mg or a flat dose of about 460 mg or a flat dose of about 480 mg or a flat dose of about 500 mg or a flat dose of about 520 mg or a flat dose of about 540 mg or a flat dose of about 560 mg or a flat dose of about 580 mg or a flat dose of about 600 mg or a flat dose of about 620 mg or a flat dose of about 640 mg or a flat dose of about 660 mg or a flat dose of about 680 mg or a flat dose of about 700 mg or a flat dose of about 720 mg or a flat dose of about 740 mg or a flat dose of about 750 mg or a flat dose of about 760 mg or a flat dose of about 780 mg or a flat dose of about 800 mg or a flat dose of about 820 mg or a flat dose of about 840 mg or a flat dose of about 860 mg or a flat dose of about 880 mg or a flat dose of about 900 mg. In some embodiments of the methods or uses or product for uses provided herein, a platinum-based agent as described herein is administered to the subject at flat dose ranging from 50 mg to 900 mg such as at a flat dose of 50 mg or a flat dose of 60 mg or a flat dose of 70 mg or a flat dose of 80 mg or a flat dose of 90 mg or a flat dose of 100 mg or a flat dose of 120 mg or a flat dose of 140 mg or a flat dose of 160 mg or a flat dose of 180 mg or a flat dose of 200 mg or a flat dose of 220 mg or a flat dose of 240 mg or a flat dose of 260 mg or a flat dose of 280 mg or a flat dose of 300 mg or a flat dose of 320 mg or a flat dose of 340 mg or a flat dose of 360 mg or a flat dose of 380 mg or a flat dose of 400 mg or a flat dose of 420 mg or a flat dose of 440 mg or a flat dose of 460 mg or a flat dose of 480 mg or a flat dose of 500 mg or a flat dose of 520 mg or a flat dose of 540 mg or a flat dose of 560 mg or a flat dose of 580 mg or a flat dose of 600 mg or a flat dose of 620 mg or a flat dose of 640 mg or a flat dose of 660 mg or a flat dose of 680 mg or a flat dose of 700 mg or a flat dose of 720 mg or a flat dose of 740 mg or a flat dose of 750 mg or a flat dose of 760 mg or a flat dose of 780 mg or a flat dose of 800 mg or a flat dose of 820 mg or a flat dose of 840 mg or a flat dose of 860 mg or a flat dose of 880 mg or a flat dose of 900 mg. In some embodiments, the flat dose is 750 mg. In some embodiments, the flat dose is 750 mg and the platinum-based agent is carboplatin. In some embodiments, the flat dose is about 600 mg and is administered once about every 1 week. In some embodiments, the flat dose is about 600 mg and is administered once about every 2 weeks. In some embodiments, the flat dose is about 600 mg and is administered once about every 3 weeks. In some embodiments, the flat dose is about 600 mg and is administered once about every 4 weeks. In some embodiments, the flat dose is about 750 mg and is administered once about every 1 week. In some embodiments, the flat dose is about 750 mg and is administered once about every 2 weeks. In some embodiments, the flat dose is about 750 mg and is administered once about every 3 weeks. In some embodiments, the flat dose is about 750 mg and is administered once about every 4 weeks. In some embodiments, the flat dose is 600 mg and is administered once about every 1 week. In some embodiments, the flat dose is 600 mg and is administered once about every 2 weeks. In some embodiments, the flat dose is 600 mg and is administered once about every 3 weeks. In some embodiments, the flat dose is 600 mg and is administered once about every 4 weeks. In some embodiments, the flat dose is 750 mg and is administered once about every 1 week. In some embodiments, the flat dose is 750 mg and is administered once about every 2 weeks. In some embodiments, the flat dose is 750 mg and is administered once about every 3 weeks. In some embodiments, the flat dose is 750 mg and is administered once about every 4 weeks. In some embodiments, the flat dose is 750 mg and is administered once about every 3 weeks (e.g., 3 days). In some embodiments, the flat dose is 750 mg and is administered once every 3 weeks. In some embodiments, the flat dose is 750 mg and is administered once every 3 weeks and the platinum-based agent is carboplatin.

In some embodiments of the methods or uses or product for uses provided herein, a platinum-based agent as described herein and an anti-TF antibody-drug conjugate or antigen-binding fragment thereof as described herein are administered to the subject at a fixed dose. In some embodiments, the fixed dose is based on the amount (e.g., mg) of the agents. In certain embodiments, the fixed dose is based on the concentration (e.g., mg/ml) of the agents. In some embodiments, the ratio of the amount (e.g., mg) of the platinum-based agent to the amount (e.g., mg) of the anti-TF antibody-drug conjugate or antigen-binding fragment thereof is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the concentration (e.g., mg/ml) of the platinum-based agent to the concentration (e.g., mg/ml) of the anti-TF antibody-drug conjugate or antigen-binding fragment thereof is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the amount (e.g., mg) of the platinum-based agent to the amount (e.g., mg) of the anti-TF antibody-drug conjugate or antigen-binding fragment thereof is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1. In some embodiments, the ratio of the concentration (e.g., mg/ml) of the platinum-based agent to the concentration (e.g., mg/ml) of the anti-TF antibody-drug conjugate or antigen-binding fragment thereof is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1.

In some embodiments, the dose of the anti-TF antibody-drug conjugate is 2.0 mg/kg and is administered once about every 3 weeks (e.g., ±3 days) and the dose of the platinum-based agent is AUC=5 and is administered once about every 3 weeks (e.g., 3 days). In some embodiments, the dose of the anti-TF antibody-drug conjugate is 2.0 mg/kg and is administered once every 3 weeks and the dose of the platinum-based agent is AUC=5 and is administered once every 3 weeks. In some embodiments, the dose of the anti-TF antibody-drug conjugate is 2.0 mg/kg and is administered once every 3 weeks and the antibody-drug conjugate is tisotumab vedotin and the dose of the platinum-based agent is AUC=5 and is administered once every 3 weeks and the platinum-based agent is carboplatin. In some embodiments, the dose of the anti-TF antibody-drug conjugate is 2.0 mg/kg and is administered once every 3 weeks and the antibody-drug conjugate is tisotumab vedotin and the dose of the platinum-based agent is AUC=5 and is administered once every 3 weeks and the platinum-based agent is cisplatin. In some embodiments, the antibody-drug conjugate and the platinum-based agent are provided as adjuvant therapy following radiation and/or surgery.

In some embodiments, the dose of the anti-TF antibody-drug conjugate is 1.3 mg/kg and is administered once about every 3 weeks (e.g., ±3 days) and the dose of the platinum-based agent is AUC=5 and is administered once about every 3 weeks (e.g., 3 days). In some embodiments, the dose of the anti-TF antibody-drug conjugate is 1.3 mg/kg and is administered once every 3 weeks and the dose of the platinum-based agent is AUC=5 and is administered once every 3 weeks. In some embodiments, the dose of the anti-TF antibody-drug conjugate is 1.3 mg/kg and is administered once every 3 weeks and the antibody-drug conjugate is tisotumab vedotin and the dose of the platinum-based agent is AUC=5 and is administered once every 3 weeks and the platinum-based agent is carboplatin. In some embodiments, the dose of the anti-TF antibody-drug conjugate is 1.3 mg/kg and is administered once every 3 weeks and the antibody-drug conjugate is tisotumab vedotin and the dose of the platinum-based agent is AUC=5 and is administered once every 3 weeks and the platinum-based agent is cisplatin. In some embodiments, the antibody-drug conjugate and the platinum-based agent are provided as adjuvant therapy following radiation and/or surgery.

In some embodiments, the dose of the anti-TF antibody-drug conjugate is 1.7 mg/kg and is administered once about every 2 weeks (e.g., ±3 days) and the dose of the platinum-based agent is AUC=5 and is administered once about every 3 weeks (e.g., ±3 days). In some embodiments, the dose of the anti-TF antibody-drug conjugate is 1.7 mg/kg and is administered once every 2 weeks and the dose of the platinum-based agent is AUC=5 and is administered once every 3 weeks. In some embodiments, the dose of the anti-TF antibody-drug conjugate is 1.7 mg/kg and is administered once every 2 weeks and the antibody-drug conjugate is tisotumab vedotin and the dose of the platinum-based agent is AUC=5 and is administered once every 3 weeks and the platinum-based agent is carboplatin. In some embodiments, the dose of the anti-TF antibody-drug conjugate is 1.7 mg/kg and is administered once every 2 weeks and the antibody-drug conjugate is tisotumab vedotin and the dose of the platinum-based agent is AUC=5 and is administered once every 3 weeks and the platinum-based agent is cisplatin. In some embodiments, the antibody-drug conjugate and the platinum-based agent are provided as adjuvant therapy following radiation and/or surgery.

In some embodiments, an anti-TF antibody-drug conjugate or antigen-binding fragment thereof as described herein and a platinum-based agent as described herein are coadministered. In some embodiments the coadministration is simultaneous or sequential. In some embodiments, an anti-TF antibody-drug conjugate as described herein is administered simultaneously with a platinum-based agent as described herein. In some embodiments, simultaneous means that the anti-TF antibody-drug conjugate and the platinum-based agent are administered to the subject less than one hour apart, such as less than about 30 minutes apart, less than about 15 minutes apart, less than about 10 minutes apart or less than about 5 minutes apart. In some embodiments, an anti-TF antibody-drug conjugate as described herein is administered sequentially with a platinum-based agent as described herein. In some embodiments, sequential administration means that the anti-TF antibody-drug conjugate and the platinum-based agent are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

The methods of treatment described herein may be, in various embodiments, neoadjuvant or adjuvant treatments. A “neoadjuvant” treatment is a treatment given as a first treatment before a main treatment, for example, to shrink a tumor before a surgical intervention. An “adjuvant” treatment is an additional treatment given after a main treatment, for example, to reduce the risk that a cancer will recur.

In some embodiments, the methods comprise administering the antibody-drug conjugate as a neoadjuvant therapy. In some embodiments, the methods comprise administering the radiation therapy as a neoadjuvant therapy. In some embodiments, the methods comprise administering the antibody-drug conjugate and the radiation therapy as a neoadjuvant therapy. In some embodiments, the methods comprise administering the antibody-drug conjugate and a chemoradiation therapy as a neoadjuvant therapy. In some embodiments, the antibody-drug conjugate and the radiation therapy are administered to a subject in need thereof who has not received previous treatment for the cancer. In some embodiments, the antibody-drug conjugate and the chemoradiation therapy are administered to a subject in need thereof who has not received previous treatment for the cancer. In some embodiments, the antibody-drug conjugate and the radiation therapy are administered before a surgical intervention for the cancer. In some embodiments, the antibody-drug conjugate and the chemoradiation therapy are administered before a surgical intervention for the cancer. In some embodiments, the surgical intervention comprises the surgical removal of one or more tumors associated with the cancer. In some embodiments, the methods comprise administering tisotumab vedotin and a chemoradiation therapy, wherein the chemotherapy is cisplatin, as a neoadjuvant therapy. In some embodiments, the methods comprise administering tisotumab vedotin and a chemoradiation therapy wherein the chemotherapy is carboplatin as a neoadjuvant therapy. In some embodiments of any of the preceding embodiments, chemoradiation is a combination of chemotherapy (such as a platinum-based agent, such as cisplatin or carboplatin) and a radiation therapy (as otherwise described herein).

In some embodiments, the methods comprise administering the antibody-drug conjugate as an adjuvant therapy. In some embodiments, the methods comprise administering the radiation therapy as an adjuvant therapy. In some embodiments, the methods comprise administering the antibody-drug conjugate and the radiation therapy as an adjuvant therapy. In some embodiments, the antibody-drug conjugate and the radiation therapy are administered after a surgical intervention for the cancer. In some embodiments, the methods comprise administering the antibody-drug conjugate and a chemoradiation therapy as an adjuvant therapy. In some embodiments, the antibody-drug conjugate and the chemoradiation therapy are administered to a subject in need thereof who has not received previous treatment for the cancer. In some embodiments, the surgical intervention comprises surgical removal of one or more tumors associated with the cancer. In some embodiments, the methods comprise administering tisotumab vedotin and a chemoradiation therapy, wherein the chemotherapy is cisplatin, as an adjuvant therapy. In some embodiments, the methods comprise administering tisotumab vedotin and a chemoradiation therapy wherein the chemotherapy is carboplatin as an adjuvant therapy. In some embodiments of any of the preceding embodiments, chemoradiation is a combination of chemotherapy (such as a platinum-based agent, such as cisplatin or carboplatin) and a radiation therapy (as otherwise described herein).

In some embodiments, a method of treatment or use or product for use described herein further comprises the administration of one or more additional therapeutic agents. In some embodiments, the one or more additional therapeutic agents are administered simultaneously with an anti-TF antibody-drug conjugate or antigen-binding fragment thereof as described herein, such as tisotumab vedotin, and a platinum-based agent as described herein, such as carboplatin or cisplatin. In some embodiments, the one or more additional therapeutic agents and an anti-TF antibody-drug conjugate or antigen-binding fragment thereof as described herein, such as tisotumab vedotin, and a platinum-based agent as described herein, such as carboplatin or cisplatin, are administered sequentially and followed or preceded by radiation therapy. In some embodiments, a method of treatment or use or product for use described herein comprises treatment with an anti-TF antibody-drug conjugate as described herein, such as tisotumab vedotin, in combination with a platinum-based agent, such as cisplatin or carboplatin, and further combined with a radiation therapy.

### VIII. Treatment Outcomes

In one aspect, a method of treating cancer with an anti-TF antibody-drug conjugate or antigen-binding fragment thereof as described herein, such as e.g, tisotumab vedotin, and a radiation therapy as described herein, wherein in some embodiments the treatment further comprises an additional chemotherapeutic agent such as a platinum-based agent, results in an improvement in one or more therapeutic effects in the subject after administration of the antibody-drug conjugate and radiation therapy. In some embodiments, the one or more therapeutic effects is the size of the tumor derived from the cancer, the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an anti-TF antibody-drug conjugate or antigen-binding fragment thereof as described herein, such as e.g., tisotumab vedotin, and a radiation therapy as described herein, may include the following criteria (RECIST Criteria 1.1):

| | Category | Criteria |
|---|---|---|
| Based on | Complete | Disappearance of all target lesions. Any |
| target lesions | Response (CR) | pathological lymph nodes must have reduction in |
| | | short axis to <10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs |
| | Disease (PD) | of target lesions, taking as reference the smallest |
| | | sum of the target LDs recorded while in trial or the |
| | | appearance of one or more new lesions. |
| Based on non- | CR | Disappearance of all non-target lesions and |
| target lesions | | normalization of tumor marker level. All lymph |
| | | nodes must be non-pathological in size (<10 mm |
| | | short axis). |
| | SD | Persistence of one or more non-target lesion(s) |
| | | or/and maintenance of tumor marker level above the |
| | | normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with an anti-TF antibody-drug conjugate or antigen-binding fragment thereof described herein, such as e.g., tisotumab vedotin, and a radiation therapy, is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-TF antibody-drug conjugate or antigen-binding fragment thereof described herein, such as e.g., tisotumab vedotin, and a radiation therapy, is assessed by measuring the size of a tumor derived from the cancer. In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate and/or the radiation therapy. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate and/or the radiation therapy. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of the tumor derived from the cancer is reduced relative to the size of the tumor before administration of the anti-TF antibody drug conjugate and the radiation therapy. In some embodiments, the size of the tumor derived from the cancer is reduced relative to the size of the tumor before administration of the anti-TF antibody drug conjugate. In some embodiments, the size of the tumor derived from the cancer is reduced relative to the size of the tumor before administration of the radiation therapy.

In one embodiment of the methods or uses or product for uses provided described herein, response to treatment with an antibody-drug conjugate or antigen-binding fragment thereof described herein, such as e.g., tisotumab vedotin, and a radiation therapy, promotes regression of a tumor derived from the cancer. In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-TF antibody-drug conjugate and/or the radiation therapy. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-TF antibody-drug conjugate and/or the radiation therapy. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, the tumor derived from the cancer regresses relative to the size of the tumor before administration of the anti-TF antibody drug conjugate and the radiation therapy. In some embodiments, the tumor derived from the cancer regresses relative to the size of the tumor before administration of the anti-TF antibody drug conjugate. In some embodiments, the tumor derived from the cancer regresses relative to the size of the tumor before administration of the radiation therapy.

In one embodiment of the methods or uses or product for uses described herein, response to treatment with an anti-TF antibody-drug conjugate or antigen-binding fragment thereof described herein, such as e.g., tisotumab vedotin, and a radiation therapy described herein, is assessed by measuring the time of progression free survival after administration of the anti-TF antibody-drug conjugate and/or the radiation therapy. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate and/or the radiation therapy. In some embodiments, the subject exhibits progression-free survival of at least about 6 months after administration of the anti-TF antibody-drug conjugate and/or the radiation therapy. In some embodiments, the subject exhibits progression-free survival of at least about one year after administration of the anti-TF antibody-drug conjugate and/or the radiation therapy. In some embodiments, the subject exhibits progression-free survival of at least about two years after administration of the anti-TF antibody-drug conjugate and/or the radiation therapy. In some embodiments, the subject exhibits progression-free survival of at least about three years after administration of the anti-TF antibody-drug conjugate and/or the radiation therapy. In some embodiments, the subject exhibits progression-free survival of at least about four years after administration of the anti-TF antibody-drug conjugate and/or the radiation therapy. In some embodiments, the subject exhibits progression-free survival of at least about five years after administration of the anti-TF antibody-drug conjugate and/or the radiation therapy. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate and/or the radiation therapy. In some embodiments, the subject exhibits progression-free survival of at least 6 months after administration of the anti-TF antibody-drug conjugate and/or the radiation therapy. In some embodiments, the subject exhibits progression-free survival of at least one year after administration of the anti-TF antibody-drug conjugate and/or the radiation therapy. In some embodiments, the subject exhibits progression-free survival of at least two years after administration of the anti-TF antibody-drug conjugate and/or the radiation therapy. In some embodiments, the subject exhibits progression-free survival of at least three years after administration of the anti-TF antibody-drug conjugate and/or the radiation therapy. In some embodiments, the subject exhibits progression-free survival of at least four years after administration of the anti-TF antibody-drug conjugate and/or the radiation therapy. In some embodiments, the subject exhibits progression-free survival of at least five years after administration of the anti-TF antibody-drug conjugate and/or the radiation therapy. In some embodiments, response to treatment is assessed by measuring the time of progression free survival after administration of the anti-TF antibody-drug conjugate and the radiation therapy. In some embodiments, response to treatment is assessed by measuring the time of progression free survival after administration of the anti-TF antibody-drug conjugate. In some embodiments, response to treatment is assessed by measuring the time of progression free survival after administration of the radiation therapy.

In one embodiment of the methods or uses or product for uses described herein, response to treatment with an anti-TF antibody-drug conjugate or antigen-binding fragment thereof described herein, such as e.g., tisotumab vedotin, and a radiation therapy described herein, is assessed by measuring the time of overall survival after administration of the anti-TF antibody-drug conjugate and/or the radiation therapy. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate and/or the radiation therapy. In some embodiments, the subject exhibits overall survival of at least about 6 months after administration of the anti-TF antibody-drug conjugate and/or the radiation therapy. In some embodiments, the subject exhibits overall survival of at least about one year after administration of the anti-TF antibody-drug conjugate and/or the radiation therapy. In some embodiments, the subject exhibits overall survival of at least about two years after administration of the anti-TF antibody-drug conjugate and/or the radiation therapy. In some embodiments, the subject exhibits overall survival of at least about three years after administration of the anti-TF antibody-drug conjugate and/or the radiation therapy. In some embodiments, the subject exhibits overall survival of at least about four years after administration of the anti-TF antibody-drug conjugate and/or the radiation therapy. In some embodiments, the subject exhibits overall survival of at least about five years after administration of the anti-TF antibody-drug conjugate and/or the radiation therapy. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate and/or the radiation therapy. In some embodiments, the subject exhibits overall survival of at least 6 months after administration of the anti-TF antibody-drug conjugate and/or the radiation therapy. In some embodiments, the subject exhibits overall survival of at least one year after administration of the anti-TF antibody-drug conjugate and/or the radiation therapy. In some embodiments, the subject exhibits overall survival of at least two years after administration of the anti-TF antibody-drug conjugate and/or the radiation therapy. In some embodiments, the subject exhibits overall survival of at least three years after administration of the anti-TF antibody-drug conjugate and/or the radiation therapy. In some embodiments, the subject exhibits overall survival of at least four years after administration of the anti-TF antibody-drug conjugate and/or the radiation therapy. In some embodiments, the subject exhibits overall survival of at least five years after administration of the anti-TF antibody-drug conjugate and/or the radiation therapy. In some embodiments, response to treatment is assessed by measuring the time of overall survival after administration of the anti-TF antibody-drug conjugate and the radiation therapy. In some embodiments, response to treatment is assessed by measuring the time of overall survival after administration of the anti-TF antibody-drug conjugate. In some embodiments, response to treatment is assessed by measuring the time of overall survival after administration of the radiation therapy.

In one embodiment of the methods or uses or product for uses described herein, response to treatment with an anti-TF antibody-drug conjugate or antigen-binding fragment thereof described herein, such as e.g., tisotumab vedotin, and a radiation therapy described herein, is assessed by measuring the duration of response to the anti-TF antibody-drug conjugate and the radiation therapy after administration of the anti-TF antibody-drug conjugate and/or the radiation therapy. In some embodiments, the duration of response to the anti-TF antibody-drug conjugate and the radiation therapy is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-TF antibody-drug conjugate and/or the radiation therapy. In some embodiments, the duration of response to the anti-TF antibody-drug conjugate and the radiation therapy is at least about 6 months after administration of the antibody-drug conjugate and/or the radiation therapy. In some embodiments, the duration of response to the anti-TF antibody-drug conjugate and the radiation therapy is at least about one year after administration of the antibody-drug conjugate and/or the radiation therapy. In some embodiments, the duration of response to the anti-TF antibody-drug conjugate and the radiation therapy is at least about two years after administration of the antibody-drug conjugate and/or the radiation therapy. In some embodiments, the duration of response to the anti-TF antibody-drug conjugate and the radiation therapy is at least about three years after administration of the antibody-drug conjugate and/or the radiation therapy. In some embodiments, the duration of response to the anti-TF antibody-drug conjugate and the radiation therapy is at least about four years after administration of the antibody-drug conjugate. In some embodiments, the duration of response to the anti-TF antibody-drug conjugate and the radiation therapy is at least about five years after administration of the antibody-drug conjugate and/or the radiation therapy. In some embodiments, the duration of response to the anti-TF antibody-drug conjugate and the radiation therapy is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-TF antibody-drug conjugate and/or the radiation therapy. In some embodiments, the duration of response to the anti-TF antibody-drug conjugate and the radiation therapy is at least 6 months after administration of the antibody-drug conjugate and/or the radiation therapy. In some embodiments, the duration of response to the anti-TF antibody-drug conjugate and the radiation therapy is at least one year after administration of the antibody-drug conjugate and/or the radiation therapy. In some embodiments, the duration of response to the anti-TF antibody-drug conjugate and the radiation therapy is at least two years after administration of the antibody-drug conjugate and/or the radiation therapy. In some embodiments, the duration of response to the anti-TF antibody-drug conjugate and the radiation therapy is at least three years after administration of the antibody-drug conjugate and/or the radiation therapy. In some embodiments, the duration of response to the anti-TF antibody-drug conjugate and the radiation therapy is at least four years after administration of the antibody-drug conjugate. In some embodiments, the duration of response to the anti-TF antibody-drug conjugate and the radiation therapy is at least five years after administration of the antibody-drug conjugate and/or the radiation therapy. In some embodiments, the duration of response is measured after administration of the anti-TF antibody drug conjugate and the radiation therapy. In some embodiments, the duration of response is measured after administration of the anti-TF antibody drug conjugate. In some embodiments, the duration of response is measured after administration of the radiation therapy.

### IX. Compositions

In some aspects, also provided herein are compositions (e.g., pharmaceutical compositions and therapeutic formulations) comprising any of the anti-TF antibody-drug conjugates or antigen-binding fragments thereof described herein, such as e.g., tisotumab vedotin. The compositions are useful for methods of treating cancer in combination with a radiation therapy, or for manufacturing medicaments for use in methods of treating cancer in combination with a radiation therapy.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, omithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating cancer as described herein. In some aspects, provided herein are compositions comprising a population of antibody-drug conjugates, wherein the antibody-drug conjugates comprise a linker attached to MMAE, wherein the antibody-drug conjugate has the following structure:

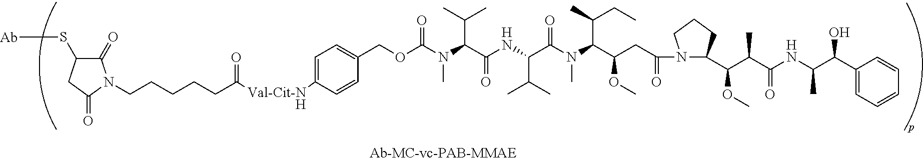

wherein p denotes a number from 1 to 8, e.g., 1, 2, 3, 4, 5, 6, 7 or 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for n.

In some embodiments, a composition comprising an anti-TF antibody-drug conjugate or antigen-binding fragment thereof as described herein, such as e.g, tisotumab vedotin, is coadministered with a composition comprising an additional chemotherapeutic agent, such as a platinum-based agent as described herein. In some embodiments the coadministration is simultaneous or sequential. In some embodiments, the anti-TF antibody-drug conjugate as described herein is administered simultaneously with the platinum-based agent. In some embodiments, simultaneous means that the anti-TF antibody-drug conjugate and the platinum-based agent are administered to the subject less than about one hour apart, such as less than about 30 minutes apart, less than about 15 minutes apart, less than about 10 minutes apart or less than about 5 minutes apart. In some embodiments, simultaneous means that the anti-TF antibody-drug conjugate and the platinum-based agent are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the platinum-based agent. In some embodiments, sequential administration means that the anti-TF antibody-drug conjugate and the platinum-based agent are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, a composition comprising an anti-TF antibody-drug conjugate as described herein and/or an platinum-based agent as described herein is coadministered with one or more therapeutic agents to eliminate or reduce the severity of one or more adverse events. In some embodiments, a composition comprising an anti-TF antibody-drug conjugate as described herein and/or an platinum-based agent as described herein is coadministered with one or more therapeutic agents to prevent the development of the adverse event or to reduce the severity of the adverse event.

In some embodiments, a composition comprising an anti-TF antibody-drug conjugate as described herein, such as e.g., tisotumab vedotin, is coadministered with one or additional therapeutic agents. In some embodiments the coadministration is simultaneous or sequential. In some embodiments, the anti-TF antibody-drug conjugate as described herein is administered simultaneously with the one or more additional therapeutic agents. In some embodiments, simultaneous means that the anti-TF antibody-drug conjugate and the one or more therapeutic agents are administered to the subject less than about one hour apart, such as less than about 30 minutes apart, less than about 15 minutes apart, less than about 10 minutes apart or less than about 5 minutes apart. In some embodiments, the anti-TF antibody-drug conjugate is administered sequentially with the one or more additional therapeutic agents. In some embodiments, simultaneous means that the anti-TF antibody-drug conjugate and the one or more therapeutic agents are administered to the subject less than one hour apart, such as less than 30 minutes apart, less than 15 minutes apart, less than 10 minutes apart or less than 5 minutes apart. In some embodiments, the anti-TF antibody-drug conjugate is administered sequentially with the one or more additional therapeutic agents. In some embodiments, sequential administration means that the anti-TF antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

### X. Articles of Manufacture and Kits

In another aspect, an article of manufacture or kit is provided which comprises an anti-TF antibody-drug conjugate described herein, such as e.g., tisotumab vedotin. The article of manufacture or kit may further comprise an additional chemotherapeutic agent, such as a platinum-based agent. The article of manufacture or kit may further comprise instructions for use of the anti-TF antibody-drug conjugate in the methods of the invention. Thus, in certain embodiments, the article of manufacture or kit comprises instructions for the use of an anti-TF antibody-drug conjugate in methods for treating cancer in a subject comprising administering to the subject an effective amount of an anti-TF antibody-drug conjugate and a radiation therapy. In some embodiments, the cancer is positive for tissue factor. In some embodiments, the cancer is a head and neck cancer, such as head and neck squamous cellular carcinoma. In some embodiments, the cancer is a gynecological cancer. In some embodiments, the gynecological cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer. In some embodiments, the gynecological cancer is an ovarian cancer. In some embodiments, the gynecological cancer is an endometrial cancer. In some embodiments, the gynecological cancer is a cervical cancer. In some embodiments, the gynecological cancer is a perineal tissue cancer. In some embodiments, the gynecological cancer is a fallopian tube cancer. In some embodiments, the cancer is a uterine cancer. In some embodiments, the gynecological cancer is a vaginal cancer. In some embodiments, the gynecological cancer is a vulvar cancer. In some embodiments, the gynecological cancer is a gestational trophoblastic disease cancer.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the second medicament is a platinum-based agent as described herein. In some embodiments, the label or package insert indicates that the first and second medicaments are to be administered sequentially or simultaneously, as described herein.

In some embodiments, the anti-TF antibody-drug conjugate is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### XI. Exemplary Embodiments

Among the embodiments provided herein are:

### A. Methods of Treatment

Embodiment 1A. A method of treating cancer in a subject, the method comprising: (i) administering to the subject a radiation therapy; and (ii) administering to the subject an antibody-drug conjugate that binds to tissue factor (TF), wherein the antibody-drug conjugate comprises an anti-TF antibody or an antigen-binding fragment thereof conjugated to an auristatin or a functional analog thereof or a functional derivative thereof.

Embodiment 2A. The method of embodiment TA, wherein the auristatin is monomethyl auristatin or a functional analog thereof or a function derivative thereof.

Embodiment 3A. The method of embodiment TA or embodiment 2A, wherein the auristatin is monomethyl auristatin E (MMAE).

Embodiment 4A. The method of any one of embodiments 1A-3A, wherein the antibody-drug conjugate is administered at a dose ranging from about 0.9 mg/kg to about 2.1 mg/kg.

Embodiment 5A. The method of any one of embodiments 1A-4A, wherein the antibody-drug conjugate is administered at a dose ranging from about 0.9 mg/kg to about 1.7 mg/kg.

Embodiment 6A. The method of embodiment 4A or embodiment 5A, wherein the antibody-drug conjugate is administered at a dose of about 1.3 mg/kg.

Embodiment 7A. The method embodiment 4A or embodiment 5A, wherein the antibody-drug conjugate is administered at a dose of about 1.7 mg/kg.

Embodiment 8A. The method of embodiment 4A, wherein the antibody-drug conjugate is administered at a dose of about 2.0 mg/kg.

Embodiment 9A. The method of any one of embodiments 1A-8A, wherein the antibody-drug conjugate is administered once about every 1 week, once about every 2 weeks, once about every 3 weeks or once about every 4 weeks.

Embodiment 10A. The method of any one of embodiments 1A-9A, wherein the antibody-drug conjugate is administered once about every 2 weeks.

Embodiment 11A. The method of any one of embodiments 1A-9A, wherein the antibody-drug conjugate is administered once about every 3 weeks.

Embodiment 12A. The method of any one of embodiments 1A-11A, wherein the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy.

Embodiment 13A. The method of any one of embodiments 1A-12A, wherein the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

Embodiment 14A. The method of any one of embodiments 1A-13A, wherein the method further comprises administering to the subject a chemotherapeutic agent.

Embodiment 15A. The method of embodiment 14A, wherein the chemotherapeutic agent is a platinum-based agent.

Embodiment 16A. The method of embodiment 15A, wherein the platinum-based agent is administered at a dose between about AUC=4 and about AUC=6.

Embodiment 17A. The method of embodiment 15A or embodiment 16A, wherein the platinum-based agent is administered a dose of about AUC=5.

Embodiment 18A. The method of any one of embodiments 15A-17A, wherein the platinum-based agent is administered once about every 1 week, once about every 2 weeks, once about every 3 weeks or once about every 4 weeks.

Embodiment 19A. The method of any one of embodiments 15A-18A, wherein the platinum-based agent is administered once about every 3 weeks.

Embodiment 20A. The method of any one of embodiments 15A-18A, wherein the platinum-based agent is administered once about every 4 weeks.

Embodiment 21A. The method of any one of embodiments 1A-20A, wherein the cancer is a solid tumor.

Embodiment 22A. The method of any one of embodiments 1A-21A, wherein the cancer is a head and neck squamous cell carcinoma.

Embodiment 23A. The method of any one of embodiments 1A-21A, wherein the cancer is a gynecological cancer.

Embodiment 24A. The method of any one of embodiments 1A-21A, wherein the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer.

Embodiment 25A. The method of any one of embodiments 1A-24A, wherein the cancer is associated with a primary tumor positive for tissue factor.

Embodiment 26A. The method of any one of embodiments 1A-25A, wherein the cancer is an early stage cancer.

Embodiment 27A. The method of embodiment 26A, wherein the cancer is a stage I or stage II cancer.

Embodiment 28A. The method of embodiment 26A or embodiment 27A, wherein the cancer is not a recurrent cancer.

Embodiment 29A. The method of any one of embodiments 26A-28A, wherein the cancer is not locally advanced.

Embodiment 30A. The method of any one of embodiments 26A-29A, wherein the cancer is not metastatic.

Embodiment 31A. The method of any one of embodiments 26A-28A, wherein the cancer is locally advanced.

Embodiment 32A. The method of any one of embodiments 1A-31A, wherein the method of treating is a neoadjuvant treatment for the cancer.

Embodiment 33A. The method of embodiment 32A, wherein the antibody-drug conjugate and radiation therapy are administered before surgical intervention for the cancer.

Embodiment 34A. The method of embodiment 32A or embodiment 33A, wherein further the platinum-based agent is administered before surgical intervention for the cancer.

Embodiment 35A. The method of embodiment 33A or embodiment 34A, wherein the antibody-drug conjugate and radiation therapy are administered before surgical removal of one or more tumors associated with the cancer.

Embodiment 36A. The method of any one of embodiments 33A-35A, wherein further the platinum-based agent is administered before surgical removal of one or more tumors associated with the cancer.

Embodiment 37A. The method of any one of embodiments 1A-36A, wherein the subject has not received prior therapy for the cancer.

Embodiment 38A. The method of any one of embodiments 1A-31A, wherein the method of treating is an adjuvant therapy for the cancer.

Embodiment 39A. The method of embodiment 38A, wherein the antibody-drug conjugate and the radiation therapy are administered after surgical intervention for the cancer.

Embodiment 40A. The method of embodiment 38A or embodiment 39A, wherein further the platinum-based agent is administered after surgical intervention for the cancer.

Embodiment 41A. The method of any one of embodiments 38A-40A, wherein the antibody-drug conjugate and radiation therapy are administered after surgical removal of one or more tumors associated with the cancer.

Embodiment 42A. The method of any one of embodiments 38A-41A, wherein further the platinum-based agent is administered after surgical removal of one or more tumors associated with the cancer.

Embodiment 43A. The method of any one of embodiments 1A-42A, wherein the anti-TF antibody or antigen-binding fragment thereof of the antibody-drug conjugate is a monoclonal antibody or a monoclonal antigen-binding fragment thereof.

Embodiment 44A. The method of any one of embodiments 1A-43A, wherein the anti-TF antibody or antigen-binding fragment thereof of the antibody-drug conjugate comprises a heavy chain variable region and a light chain variable region, wherein the heavy chain variable region comprises: (i) a CDR-H1 comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and wherein the light chain variable region comprises: (i) a CDR-L1 comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6, wherein the CDRs of the anti-TF antibody or antigen-binding fragment thereof are defined by the IMGT numbering scheme.

Embodiment 45A. The method of any one of embodiments 1A-44A, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

Embodiment 46A. The method of any one of embodiments 1A-45A, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

Embodiment 47A. The method of any one of embodiments 1A-46A, wherein the anti-TF antibody of the antibody-drug conjugate is tisotumab.

Embodiment 48A. The method of any one of embodiments 1A-47A, wherein the antibody-drug conjugate further comprises a linker between the anti-TF antibody or antigen-binding fragment thereof and the auristatin.

Embodiment 49A. The method of embodiment 48A, wherein the linker is a cleavable peptide linker.

Embodiment 50A. The method of embodiment 49A, wherein the cleavable peptide linker has a formula: -MC-vc-PAB-, wherein:
a) MC is:

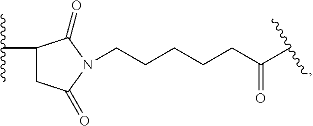 [SMILES: *C(=O)CCCCCN1C(=O)CC(*)C1=O]

b) vc is the dipeptide valine-citrulline, and
c) PAB is:

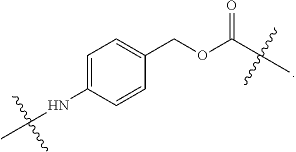 [SMILES: *Nc1ccc(COC(*)=O)cc1]

Embodiment 51A. The method of any one of embodiments 48A-50A, wherein the linker is attached to sulphydryl residues of the anti-TF antibody obtained by partial reduction or full reduction of the anti-TF antibody or antigen-binding fragment thereof.

Embodiment 52A. The method of embodiment 51A, wherein the linker is attached to MMAE, wherein the antibody-drug conjugate has the following structure:

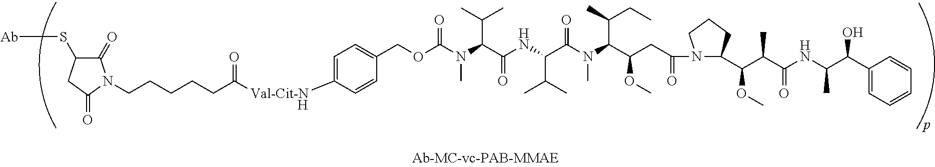

wherein p denotes a number from 1 to 8, S represents a sulphydryl residue of the anti-TF antibody, and Ab designates the anti-TF antibody or antigen-binding fragment thereof.

Embodiment 53A. The method of embodiment 52A, wherein the average value of p in a population of the antibody-drug conjugates is about 4.

Embodiment 54A. The method of any one of embodiments 1A-53A, wherein the antibody-drug conjugate is tisotumab vedotin.

Embodiment 55A. The method of any one of embodiments 1A-54A, wherein the route of administration for the antibody-drug conjugate is intravenous.

Embodiment 56A. The method of any one of embodiments 15A-55A, wherein the platinum-based agent is selected from the group consisting of carboplatin, cisplatin, oxaliplatin, and nedaplatin.

Embodiment 57A. The method of any one of embodiments 15A-56A, wherein the platinum-based agent is carboplatin.

Embodiment 58A. The method of any one of embodiments 15A-56A, wherein the platinum-based agent is cisplatin.

Embodiment 59A. The method of any one of embodiments 15A-58A, wherein the route of administration for the platinum-based agent is intravenous.

Embodiment 60A. The method of any one of embodiments 15A-59A, wherein the platinum-based agent and the antibody-drug conjugate are administered sequentially.

Embodiment 61A. The method of any one of embodiments 15A-59A, wherein the platinum-based agent and the antibody-drug conjugate are administered simultaneously.

Embodiment 62A. The method of any one of embodiments 1A-61A, wherein the subject is a human.

Embodiment 63A. The method of any one of embodiments 1A-62A, wherein the antibody-drug conjugate is in a pharmaceutical composition comprising the antibody-drug conjugate and a pharmaceutically acceptable carrier.

Embodiment 64A. The method of any one of embodiments 15A-63A, wherein the platinum-based agent is in a pharmaceutical composition comprising the platinum-based agent and a pharmaceutical acceptable carrier.

### B. Antibody-Drug Conjugate for Use

Embodiment 1B. An antibody-drug conjugate that binds TF for use in the treatment of cancer in a subject, wherein the antibody-drug conjugate is for administration, or to be administered in combination with, a radiation therapy, wherein the antibody-drug conjugate comprises an anti-TF antibody or an antigen-binding fragment thereof conjugated to an auristatin or a functional analog thereof or a functional derivative thereof.

Embodiment 2B. The antibody-drug conjugate for use of embodiment 1B, wherein the auristatin is monomethyl auristatin or a functional analog thereof or a function derivative thereof.

Embodiment 3B. The antibody-drug conjugate for use of embodiment 1B or embodiment 2B, wherein the auristatin is monomethyl auristatin E (MMAE).

Embodiment 4B. The antibody-drug conjugate for use of any one of embodiments 1B-3B, wherein the antibody-drug conjugate is administered at a dose ranging from about 0.9 mg/kg to about 2.1 mg/kg.

Embodiment 5B. The antibody-drug conjugate for use of any one of embodiments 1B-4B, wherein the antibody-drug conjugate is administered at a dose ranging from about 0.9 mg/kg to about 1.7 mg/kg.

Embodiment 6B. The antibody-drug conjugate for use of embodiment 4B or embodiment 5B, wherein the antibody-drug conjugate is administered at a dose of about 1.3 mg/kg.

Embodiment 7B. The method embodiment 4B or embodiment 5B, wherein the antibody-drug conjugate is administered at a dose of about 1.7 mg/kg.

Embodiment 8B. The antibody-drug conjugate for use of embodiment 4B, wherein the antibody-drug conjugate is administered at a dose of about 2.0 mg/kg.

Embodiment 9B. The antibody-drug conjugate for use of any one of embodiments 1B-8B, wherein the antibody-drug conjugate is administered once about every 1 week, once about every 2 weeks, once about every 3 weeks or once about every 4 weeks.

Embodiment 10B. The antibody-drug conjugate for use of any one of embodiments 1B-9B, wherein the antibody-drug conjugate is administered once about every 2 weeks.

Embodiment 11B. The antibody-drug conjugate for use of any one of embodiments 1B-9B, wherein the antibody-drug conjugate is administered once about every 3 weeks.

Embodiment 12B. The antibody-drug conjugate for use of any one of embodiments 1B-11B, wherein the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy.

Embodiment 13B. The antibody-drug conjugate for use of any one of embodiments 1B-12B, wherein the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

Embodiment 14B. The antibody-drug conjugate for use of any one of embodiments 1B-13B, wherein the method further comprises administering to the subject a chemotherapeutic agent.

Embodiment 15B. The antibody-drug conjugate for use of embodiment 14B, wherein the chemotherapeutic agent is a platinum-based agent.

Embodiment 16B. The antibody-drug conjugate for use of embodiment 15B, wherein the platinum-based agent is administered at a dose between about AUC=4 and about AUC=6.

Embodiment 17B. The antibody-drug conjugate for use of embodiment 15B or embodiment 16B, wherein the platinum-based agent is administered a dose of about AUC=5.

Embodiment 18B. The antibody-drug conjugate for use of any one of embodiments 15B-17B, wherein the platinum-based agent is administered once about every 1 week, once about every 2 weeks, once about every 3 weeks or once about every 4 weeks.

Embodiment 19B. The antibody-drug conjugate for use of any one of embodiments 15B-18B, wherein the platinum-based agent is administered once about every 3 weeks.

Embodiment 20B. The antibody-drug conjugate for use of any one of embodiments 15B-18B, wherein the platinum-based agent is administered once about every 4 weeks.

Embodiment 21B. The antibody-drug conjugate for use of any one of embodiments 1B-20B, wherein the cancer is a solid tumor.

Embodiment 22B. The antibody-drug conjugate for use of any one of embodiments 1B-21B, wherein the cancer is a head and neck squamous cell carcinoma.

Embodiment 23B. The antibody-drug conjugate for use of any one of embodiments 1B-21B, wherein the cancer is a gynecological cancer.

Embodiment 24B. The antibody-drug conjugate for use of any one of embodiments 1B-21B, wherein the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer.

Embodiment 25B. The antibody-drug conjugate for use of any one of embodiments 1B-24B, wherein the cancer is associated with a primary tumor positive for tissue factor.

Embodiment 26B. The antibody-drug conjugate for use of any one of embodiments 1B-25B, wherein the cancer is an early stage cancer.

Embodiment 27B. The antibody-drug conjugate for use of embodiment 26B, wherein the cancer is a stage I or stage II cancer.

Embodiment 28B. The antibody-drug conjugate for use of embodiment 26B or embodiment 27B, wherein the cancer is not a recurrent cancer.

Embodiment 29B. The antibody-drug conjugate for use of any one of embodiments 26B-28B, wherein the cancer is not locally advanced.

Embodiment 30B. The antibody-drug conjugate for use of any one of embodiments 26B-29B, wherein the cancer is not metastatic.

Embodiment 31B. The antibody-drug conjugate for use of any one of embodiments 26B-28B, wherein the cancer is locally advanced.

Embodiment 32B. The antibody-drug conjugate for use of any one of embodiments 1B-31B, wherein the method of treating is a neoadjuvant treatment for the cancer.

Embodiment 33B. The antibody-drug conjugate for use of embodiment 32B, wherein the antibody-drug conjugate and radiation therapy are administered before surgical intervention for the cancer.

Embodiment 34B. The antibody-drug conjugate for use of embodiment 32B or embodiment 33B, wherein further the platinum-based agent is administered before surgical intervention for the cancer.

Embodiment 35B. The antibody-drug conjugate for use of embodiment 33B or embodiment 34B, wherein the antibody-drug conjugate and radiation therapy are administered before surgical removal of one or more tumors associated with the cancer.

Embodiment 36B. The antibody-drug conjugate for use of any one of embodiments 33B-35B, wherein further the platinum-based agent is administered before surgical removal of one or more tumors associated with the cancer.

Embodiment 37B. The antibody-drug conjugate for use of any one of embodiments 1B-36B, wherein the subject has not received prior therapy for the cancer.

Embodiment 38B. The antibody-drug conjugate for use of any one of embodiments 1B-31B, wherein method of treating is an adjuvant therapy for the cancer.

Embodiment 39B. The antibody-drug conjugate for use of embodiment 38B, wherein the antibody-drug conjugate and the radiation therapy are administered after surgical intervention for the cancer.

Embodiment 40B. The antibody-drug conjugate for use of embodiment 38B or embodiment 39B, wherein further the platinum-based agent is administered after surgical intervention for the cancer.

Embodiment 41B. The antibody-drug conjugate for use of any one of embodiments 38B-40B, wherein the antibody-drug conjugate and radiation therapy are administered after surgical removal of one or more tumors associated with the cancer.

Embodiment 42B. The antibody-drug conjugate for use of any one of embodiments 38B-41B, wherein further the platinum-based agent is administered after surgical removal of one or more tumors associated with the cancer.

Embodiment 43B. The antibody-drug conjugate for use of any one of embodiments 1B-42B, wherein the anti-TF antibody or antigen-binding fragment thereof of the antibody-drug conjugate is a monoclonal antibody or a monoclonal antigen-binding fragment thereof.

Embodiment 44B. The antibody-drug conjugate for use of any one of embodiments 1B-43B, wherein the anti-TF antibody or antigen-binding fragment thereof of the antibody-drug conjugate comprises a heavy chain variable region and a light chain variable region, wherein the heavy chain variable region comprises: (i) a CDR-H1 comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and wherein the light chain variable region comprises: (i) a CDR-L1 comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6, wherein the CDRs of the anti-TF antibody or antigen-binding fragment thereof are defined by the IMGT numbering scheme.

Embodiment 45B. The antibody-drug conjugate for use of any one of embodiments 1B-44B, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

Embodiment 46B. The antibody-drug conjugate for use of any one of embodiments 1B-45B, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

Embodiment 47B. The antibody-drug conjugate for use of any one of embodiments 1B-46B, wherein the anti-TF antibody of the antibody-drug conjugate is tisotumab.

Embodiment 48B. The antibody-drug conjugate for use of any one of embodiments 1B-47B, wherein the antibody-drug conjugate further comprises a linker between the anti-TF antibody or antigen-binding fragment thereof and the auristatin.

Embodiment 49B. The antibody-drug conjugate for use of embodiment 48B, wherein the linker is a cleavable peptide linker.

Embodiment 50B. The antibody-drug conjugate for use of embodiment 49B, wherein the cleavable peptide linker has a formula: -MC-vc-PAB-, wherein:
a) MC is:

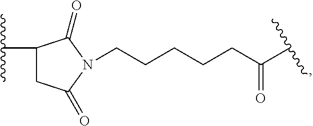 [SMILES: *C(=O)CCCCCN1C(=O)CC(*)C1=O]

b) vc is the dipeptide valine-citrulline, and
c) PAB is:

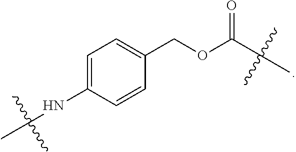 [SMILES: *Nc1ccc(COC(*)=O)cc1]

Embodiment 51B. The antibody-drug conjugate for use of any one of embodiments 48B-50B, wherein the linker is attached to sulphydryl residues of the anti-TF antibody obtained by partial reduction or full reduction of the anti-TF antibody or antigen-binding fragment thereof.

Embodiment 52B. The antibody-drug conjugate for use of embodiment 51B, wherein the linker is attached to MMAE, wherein the antibody-drug conjugate has the following structure:

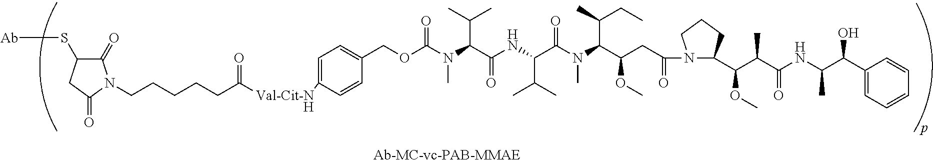

wherein p denotes a number from 1 to 8, S represents a sulphydryl residue of the anti-TF antibody, and Ab designates the anti-TF antibody or antigen-binding fragment thereof.

Embodiment 53B. The antibody-drug conjugate for use of embodiment 52B, wherein the average value of p in a population of the antibody-drug conjugates is about 4.

Embodiment 54B. The antibody-drug conjugate for use of any one of embodiments 1B-53B, wherein the antibody-drug conjugate is tisotumab vedotin.

Embodiment 55B. The antibody-drug conjugate for use of any one of embodiments 1B-54B, wherein the route of administration for the antibody-drug conjugate is intravenous.

Embodiment 56B. The antibody-drug conjugate for use of any one of embodiments 15B-55B, wherein the platinum-based agent is selected from the group consisting of carboplatin, cisplatin, oxaliplatin, and nedaplatin.

Embodiment 57B. The antibody-drug conjugate for use of any one of embodiments 15B-56B, wherein the platinum-based agent is carboplatin.

Embodiment 58B. The antibody-drug conjugate for use of any one of embodiments 15B-56B, wherein the platinum-based agent is cisplatin.

Embodiment 59B. The antibody-drug conjugate for use of any one of embodiments 15B-58B, wherein the route of administration for the platinum-based agent is intravenous.

Embodiment 60B. The antibody-drug conjugate for use of any one of embodiments 15B-59B, wherein the platinum-based agent and the antibody-drug conjugate are administered sequentially.

Embodiment 61B. The antibody-drug conjugate for use of any one of embodiments 15B-59B, wherein the platinum-based agent and the antibody-drug conjugate are administered simultaneously.

Embodiment 62B. The antibody-drug conjugate for use of any one of embodiments 1B-61B, wherein the subject is a human.

Embodiment 63B. The antibody-drug conjugate for use of any one of embodiments 1B-62B, wherein the antibody-drug conjugate is in a pharmaceutical composition comprising the antibody-drug conjugate and a pharmaceutically acceptable carrier.

Embodiment 64B. The antibody-drug conjugate for use of any one of embodiments 15B-63B, wherein the platinum-based agent is in a pharmaceutical composition comprising the platinum-based agent and a pharmaceutical acceptable carrier.

### C. Use of an Antibody-Drug Conjugate

Embodiment 1C. Use of an antibody-drug conjugate that binds TF for the manufacture of a medicament for treating cancer in a subject, wherein the medicament is for use in combination with a radiation therapy, wherein the antibody-drug conjugate comprises an anti-TF antibody or an antigen-binding fragment thereof conjugated to an auristatin or a functional analog thereof or a functional derivative thereof.

Embodiment 2C. The use of embodiment 1C, wherein the auristatin is monomethyl auristatin or a functional analog thereof or a function derivative thereof.

Embodiment 3C. The use of embodiment 1C or embodiment 2C, wherein the auristatin is monomethyl auristatin E (MMAE).

Embodiment 4C. The use of any one of embodiments 1C-3C, wherein the antibody-drug conjugate is administered at a dose ranging from about 0.9 mg/kg to about 2.1 mg/kg.

Embodiment 5C. The use of any one of embodiments 1C-4C, wherein the antibody-drug conjugate is administered at a dose ranging from about 0.9 mg/kg to about 1.7 mg/kg.

Embodiment 6C. The use of embodiment 4C or embodiment 5C, wherein the antibody-drug conjugate is administered at a dose of about 1.3 mg/kg.

Embodiment 7C. The method embodiment 4C or embodiment 5C, wherein the antibody-drug conjugate is administered at a dose of about 1.7 mg/kg.

Embodiment 8C. The use of embodiment 4C, wherein the antibody-drug conjugate is administered at a dose of about 2.0 mg/kg.

Embodiment 9C. The use of any one of embodiments 1C-8C, wherein the antibody-drug conjugate is administered once about every 1 week, once about every 2 weeks, once about every 3 weeks or once about every 4 weeks.

Embodiment 10C. The use of any one of embodiments 1C-9C, wherein the antibody-drug conjugate is administered once about every 2 weeks.

Embodiment 11C. The use of any one of embodiments 1C-9C, wherein the antibody-drug conjugate is administered once about every 3 weeks.

Embodiment 12C. The use of any one of embodiments 1C-11C, wherein the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy.

Embodiment 13C. The use of any one of embodiments 1C-12C, wherein the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

Embodiment 14C. The use of any one of embodiments 1C-13C, wherein the method further comprises administering to the subject a chemotherapeutic agent.

Embodiment 15C. The use of embodiment 14C, wherein the chemotherapeutic agent is a platinum-based agent.

Embodiment 16C. The use of embodiment 15C, wherein the platinum-based agent is administered at a dose between about AUC=4 and about AUC=6.

Embodiment 17C. The use of embodiment 15C or embodiment 16C, wherein the platinum-based agent is administered a dose of about AUC=5.

Embodiment 18C. The use of any one of embodiments 15C-17C, wherein the platinum-based agent is administered once about every 1 week, once about every 2 weeks, once about every 3 weeks or once about every 4 weeks.

Embodiment 19C. The use of any one of embodiments 15C-18C, wherein the platinum-based agent is administered once about every 3 weeks.

Embodiment 20C. The use of any one of embodiments 15C-18C, wherein the platinum-based agent is administered once about every 4 weeks.

Embodiment 21C. The use of any one of embodiments 1C-20C, wherein the cancer is a solid tumor.

Embodiment 22C. The use of any one of embodiments 1C-21C, wherein the cancer is a head and neck squamous cell carcinoma.

Embodiment 23C. The use of any one of embodiments 1C-21C, wherein the cancer is a gynecological cancer.

Embodiment 24C. The use of any one of embodiments 1C-21C, wherein the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer.

Embodiment 25C. The use of any one of embodiments 1C-24C, wherein the cancer is associated with a primary tumor positive for tissue factor.

Embodiment 26C. The use of any one of embodiments 1C-25C, wherein the cancer is an early stage cancer.

Embodiment 27C. The use of embodiment 26C, wherein the cancer is a stage I or stage II cancer.

Embodiment 28C. The use of embodiment 26C or embodiment 27C, wherein the cancer is not a recurrent cancer.

Embodiment 29C. The use of any one of embodiments 26C-28C, wherein the cancer is not locally advanced.

Embodiment 30C. The use of any one of embodiments 26C-29C, wherein the cancer is not metastatic.

Embodiment 31C. The use of any one of embodiments 26C-28C, wherein the cancer is locally advanced.

Embodiment 32C. The use of any one of embodiments 1C-31C, wherein the method of treating is a neoadjuvant treatment for the cancer.

Embodiment 33C. The use of embodiment 32C, wherein the antibody-drug conjugate and radiation therapy are administered before surgical intervention for the cancer.

Embodiment 34C. The use of embodiment 32C or embodiment 33C, wherein further the platinum-based agent is administered before surgical intervention for the cancer.

Embodiment 35C. The use of embodiment 33C or embodiment 34C, wherein the antibody-drug conjugate and radiation therapy are administered before surgical removal of one or more tumors associated with the cancer.

Embodiment 36C. The use of any one of embodiments 33C-35C, wherein further the platinum-based agent is administered before surgical removal of one or more tumors associated with the cancer.

Embodiment 37C. The use of any one of embodiments 1C-36C, wherein the subject has not received prior therapy for the cancer.

Embodiment 38C. The use of any one of embodiments 1C-31C, wherein method of treating is an adjuvant therapy for the cancer.

Embodiment 39C. The use of embodiment 38C, wherein the antibody-drug conjugate and the radiation therapy are administered after surgical intervention for the cancer.

Embodiment 40C. The use of embodiment 38C or embodiment 39C, wherein further the platinum-based agent is administered after surgical intervention for the cancer.

Embodiment 41C. The use of any one of embodiments 38C-40C, wherein the antibody-drug conjugate and radiation therapy are administered after surgical removal of one or more tumors associated with the cancer.

Embodiment 42C. The use of any one of embodiments 38C-41C, wherein further the platinum-based agent is administered after surgical removal of one or more tumors associated with the cancer.

Embodiment 43C. The use of any one of embodiments 1C-42C, wherein the anti-TF antibody or antigen-binding fragment thereof of the antibody-drug conjugate is a monoclonal antibody or a monoclonal antigen-binding fragment thereof.

Embodiment 44C. The use of any one of embodiments 1C-43C, wherein the anti-TF antibody or antigen-binding fragment thereof of the antibody-drug conjugate comprises a heavy chain variable region and a light chain variable region, wherein the heavy chain variable region comprises: (i) a CDR-H1 comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and wherein the light chain variable region comprises: (i) a CDR-L1 comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6, wherein the CDRs of the anti-TF antibody or antigen-binding fragment thereof are defined by the IMGT numbering scheme.

Embodiment 45C. The use of any one of embodiments 1C-44C, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO: 8.

Embodiment 46C. The use of any one of embodiments 1C-45C, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

Embodiment 47C. The use of any one of embodiments 1C-46C, wherein the anti-TF antibody of the antibody-drug conjugate is tisotumab.

Embodiment 48C. The use of any one of embodiments 1C-47C, wherein the antibody-drug conjugate further comprises a linker between the anti-TF antibody or antigen-binding fragment thereof and the auristatin.

Embodiment 49C. The use of embodiment 48C, wherein the linker is a cleavable peptide linker.

Embodiment 50C. The use of embodiment 49C, wherein the cleavable peptide linker has a formula: -MC-vc-PAB-, wherein:
a) MC is:

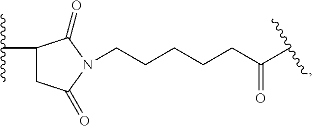 [SMILES: *C(=O)CCCCCN1C(=O)CC(*)C1=O]

b) vc is the dipeptide valine-citrulline, and
c) PAB is:

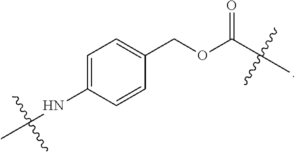 [SMILES: *Nc1ccc(COC(*)=O)cc1]

Embodiment 51C. The use of any one of embodiments 48C-50C, wherein the linker is attached to sulphydryl residues of the anti-TF antibody obtained by partial reduction or full reduction of the anti-TF antibody or antigen-binding fragment thereof.

Embodiment 52C. The use of embodiment 51C, wherein the linker is attached to MMAE, wherein the antibody-drug conjugate has the following structure:

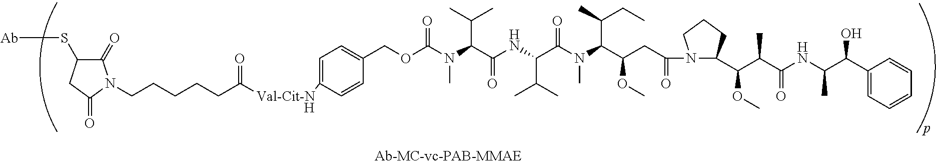

wherein p denotes a number from 1 to 8, S represents a sulphydryl residue of the anti-TF antibody, and Ab designates the anti-TF antibody or antigen-binding fragment thereof.

Embodiment 53C. The use of embodiment 52C, wherein the average value of p in a population of the antibody-drug conjugates is about 4.

Embodiment 54C. The use of any one of embodiments 1C-53C, wherein the antibody-drug conjugate is tisotumab vedotin.

Embodiment 55C. The use of any one of embodiments 1C-54C, wherein the route of administration for the antibody-drug conjugate is intravenous.

Embodiment 56C. The use of any one of embodiments 15C-55C, wherein the platinum-based agent is selected from the group consisting of carboplatin, cisplatin, oxaliplatin, and nedaplatin.

Embodiment 57C. The use of any one of embodiments 15C-56C, wherein the platinum-based agent is carboplatin.

Embodiment 58C. The use of any one of embodiments 15C-56C, wherein the platinum-based agent is cisplatin.

Embodiment 59C. The use of any one of embodiments 15C-58C, wherein the route of administration for the platinum-based agent is intravenous.

Embodiment 60C. The use of any one of embodiments 15C-59C, wherein the platinum-based agent and the antibody-drug conjugate are administered sequentially.

Embodiment 61C. The use of any one of embodiments 15C-59C, wherein the platinum-based agent and the antibody-drug conjugate are administered simultaneously.

Embodiment 62C. The use of any one of embodiments 1C-61C, wherein the subject is a human.

Embodiment 63C. The use of any one of embodiments 1C-62C, wherein the antibody-drug conjugate is in a pharmaceutical composition comprising the antibody-drug conjugate and a pharmaceutically acceptable carrier.

Embodiment 64C. The use of any one of embodiments 15C-63C, wherein the platinum-based agent is in a pharmaceutical composition comprising the platinum-based agent and a pharmaceutical acceptable carrier.

### D. Use of an Antibody-Drug Conjugate.

Embodiment 1D. Use of an antibody-drug conjugate that binds TF for treating cancer in a subject, wherein the use is in combination with a radiation therapy, wherein the antibody-drug conjugate comprises an anti-TF antibody or an antigen-binding fragment thereof conjugated to an auristatin or a functional analog thereof or a functional derivative thereof.

Embodiment 2D. The use of embodiment 1D, wherein the auristatin is monomethyl auristatin or a functional analog thereof or a function derivative thereof.

Embodiment 3D. The use of embodiment 1D or embodiment 2D, wherein the auristatin is monomethyl auristatin E (MMAE).

Embodiment 4D. The use of any one of embodiments 1D-3D, wherein the antibody-drug conjugate is administered at a dose ranging from about 0.9 mg/kg to about 2.1 mg/kg.

Embodiment 5D. The use of any one of embodiments 1D-4D, wherein the antibody-drug conjugate is administered at a dose ranging from about 0.9 mg/kg to about 1.7 mg/kg.

Embodiment 6D. The use of embodiment 4D or embodiment 5D, wherein the antibody-drug conjugate is administered at a dose of about 1.3 mg/kg.

Embodiment 7D. The method embodiment 4D or embodiment 5D, wherein the antibody-drug conjugate is administered at a dose of about 1.7 mg/kg.

Embodiment 8D. The use of embodiment 4D, wherein the antibody-drug conjugate is administered at a dose of about 2.0 mg/kg.

Embodiment 9D. The use of any one of embodiments 1D-8D, wherein the antibody-drug conjugate is administered once about every 1 week, once about every 2 weeks, once about every 3 weeks or once about every 4 weeks.

Embodiment 10D. The use of any one of embodiments 1D-9D, wherein the antibody-drug conjugate is administered once about every 2 weeks.

Embodiment 11D. The use of any one of embodiments 1D-9D, wherein the antibody-drug conjugate is administered once about every 3 weeks.

Embodiment 12D. The use of any one of embodiments 1D-11D, wherein the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy.

Embodiment 13D. The use of any one of embodiments 1D-12D, wherein the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

Embodiment 14D. The use of any one of embodiments 1D-13D, wherein the method further comprises administering to the subject a chemotherapeutic agent.

Embodiment 15D. The use of embodiment 14D, wherein the chemotherapeutic agent is a platinum-based agent.

Embodiment 16D. The use of embodiment 15D, wherein the platinum-based agent is administered at a dose between about AUC=4 and about AUC=6.

Embodiment 17D. The use of embodiment 15D or embodiment 16D, wherein the platinum-based agent is administered a dose of about AUC=5.

Embodiment 18D. The use of any one of embodiments 15D-17D, wherein the platinum-based agent is administered once about every 1 week, once about every 2 weeks, once about every 3 weeks or once about every 4 weeks.

Embodiment 19D. The use of any one of embodiments 15D-18D, wherein the platinum-based agent is administered once about every 3 weeks.

Embodiment 20D. The use of any one of embodiments 15D-18D, wherein the platinum-based agent is administered once about every 4 weeks.

Embodiment 21D. The use of any one of embodiments 1D-20D, wherein the cancer is a solid tumor.

Embodiment 22D. The use of any one of embodiments 1D-21D, wherein the cancer is a head and neck squamous cell carcinoma.

Embodiment 23D. The use of any one of embodiments 1D-21D, wherein the cancer is a gynecological cancer.

Embodiment 24D. The use of any one of embodiments 1D-21D, wherein the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer.

Embodiment 25D. The use of any one of embodiments 1D-24D, wherein the cancer is associated with a primary tumor positive for tissue factor.

Embodiment 26D. The use of any one of embodiments 1D-25D, wherein the cancer is an early stage cancer.

Embodiment 27D. The use of embodiment 26D, wherein the cancer is a stage I or stage II cancer.

Embodiment 28D. The use of embodiment 26D or embodiment 27D, wherein the cancer is not a recurrent cancer.

Embodiment 29D. The use of any one of embodiments 26D-28D, wherein the cancer is not locally advanced.

Embodiment 30D. The use of any one of embodiments 26D-29D, wherein the cancer is not metastatic.

Embodiment 31D. The use of any one of embodiments 26D-28D, wherein the cancer is locally advanced.

Embodiment 32D. The use of any one of embodiments 1D-31D, wherein the method of treating is a neoadjuvant treatment for the cancer.

Embodiment 33D. The use of embodiment 32D, wherein the antibody-drug conjugate and radiation therapy are administered before surgical intervention for the cancer.

Embodiment 34D. The use of embodiment 32D or embodiment 33D, wherein further the platinum-based agent is administered before surgical intervention for the cancer.

Embodiment 35D. The use of embodiment 33D or embodiment 34D, wherein the antibody-drug conjugate and radiation therapy are administered before surgical removal of one or more tumors associated with the cancer.

Embodiment 36D. The use of any one of embodiments 33D-35D, wherein further the platinum-based agent is administered before surgical removal of one or more tumors associated with the cancer.

Embodiment 37D. The use of any one of embodiments 1D-36D, wherein the subject has not received prior therapy for the cancer.

Embodiment 38D. The use of any one of embodiments 1D-31D, wherein method of treating is an adjuvant therapy for the cancer.

Embodiment 39D. The use of embodiment 38D, wherein the antibody-drug conjugate and the radiation therapy are administered after surgical intervention for the cancer.

Embodiment 40D. The use of embodiment 38D or embodiment 39D, wherein further the platinum-based agent is administered after surgical intervention for the cancer.

Embodiment 41D. The use of any one of embodiments 38D-40D, wherein the antibody-drug conjugate and radiation therapy are administered after surgical removal of one or more tumors associated with the cancer.

Embodiment 42D. The use of any one of embodiments 38D-41D, wherein further the platinum-based agent is administered after surgical removal of one or more tumors associated with the cancer.

Embodiment 43D. The use of any one of embodiments 1D-42D, wherein the anti-TF antibody or antigen-binding fragment thereof of the antibody-drug conjugate is a monoclonal antibody or a monoclonal antigen-binding fragment thereof.

Embodiment 44D. The use of any one of embodiments 1D-43D, wherein the anti-TF antibody or antigen-binding fragment thereof of the antibody-drug conjugate comprises a heavy chain variable region and a light chain variable region, wherein the heavy chain variable region comprises: (i) a CDR-H1 comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and wherein the light chain variable region comprises: (i) a CDR-L1 comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6, wherein the CDRs of the anti-TF antibody or antigen-binding fragment thereof are defined by the IMGT numbering scheme.

Embodiment 45D. The use of any one of embodiments 1D-44D, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

Embodiment 46D. The use of any one of embodiments 1D-45D, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

Embodiment 47D. The use of any one of embodiments 1D-46D, wherein the anti-TF antibody of the antibody-drug conjugate is tisotumab.

Embodiment 48D. The use of any one of embodiments 1D-47D, wherein the antibody-drug conjugate further comprises a linker between the anti-TF antibody or antigen-binding fragment thereof and the auristatin.

Embodiment 49D. The use of embodiment 48D, wherein the linker is a cleavable peptide linker.

Embodiment 50D. The use of embodiment 49D, wherein the cleavable peptide linker has a formula: -MC-vc-PAB-, wherein:
a) MC is:

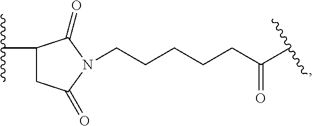 [SMILES: *C(=O)CCCCCN1C(=O)CC(*)C1=O]

b) vc is the dipeptide valine-citrulline, and
c) PAB is:

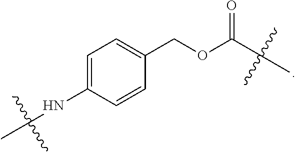 [SMILES: *Nc1ccc(COC(*)=O)cc1]

Embodiment 51D. The use of any one of embodiments 48D-50D, wherein the linker is attached to sulphydryl residues of the anti-TF antibody obtained by partial reduction or full reduction of the anti-TF antibody or antigen-binding fragment thereof.

Embodiment 52D. The use of embodiment 51D, wherein the linker is attached to MMAE, wherein the antibody-drug conjugate has the following structure:

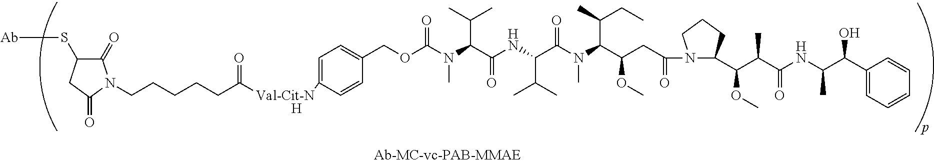

wherein p denotes a number from 1 to 8, S represents a sulphydryl residue of the anti-TF antibody, and Ab designates the anti-TF antibody or antigen-binding fragment thereof.

Embodiment 53D. The use of embodiment 52D, wherein the average value of p in a population of the antibody-drug conjugates is about 4.

Embodiment 54D. The use of any one of embodiments 1D-53D, wherein the antibody-drug conjugate is tisotumab vedotin.

Embodiment 55D. The use of any one of embodiments 1D-54D, wherein the route of administration for the antibody-drug conjugate is intravenous.

Embodiment 56D. The use of any one of embodiments 15D-55D, wherein the platinum-based agent is selected from the group consisting of carboplatin, cisplatin, oxaliplatin, and nedaplatin.

Embodiment 57D. The use of any one of embodiments 15D-56D, wherein the platinum-based agent is carboplatin.

Embodiment 58D. The use of any one of embodiments 15D-56D, wherein the platinum-based agent is cisplatin.

Embodiment 59D. The use of any one of embodiments 15D-58D, wherein the route of administration for the platinum-based agent is intravenous.

Embodiment 60D. The use of any one of embodiments 15D-59D, wherein the platinum-based agent and the antibody-drug conjugate are administered sequentially.

Embodiment 61D. The use of any one of embodiments 15D-59D, wherein the platinum-based agent and the antibody-drug conjugate are administered simultaneously.

Embodiment 62D. The use of any one of embodiments 1D-61D, wherein the subject is a human.

Embodiment 63D. The use of any one of embodiments 1D-62D, wherein the antibody-drug conjugate is in a pharmaceutical composition comprising the antibody-drug conjugate and a pharmaceutically acceptable carrier.

Embodiment 64D. The use of any one of embodiments 15D-63D, wherein the platinum-based agent is in a pharmaceutical composition comprising the platinum-based agent and a pharmaceutical acceptable carrier.

### EXAMPLES

The application may be better understood by reference to the following non-limiting examples, which are provided as exemplary embodiments of the application. The following examples are presented in order to more fully illustrate embodiments and should in no way be construed as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1a: Elevated CD142/Tissue Factor Expression is Associated with Samples Derived from Head and Neck Squamous Cell Carcinomas

CD142, also known as Tissue Factor (TF), is a protein that is expressed in solid tumors. Tumors found in HNSCC patients are a solid tumor type that arises from the squamous epithelial cells lining the upper aerodigestive tract. In this Example, experiments were performed to demonstrate that many solid tumors samples collected from HNSCC patients showed TF expression, as analyzed by immunohistochemistry (IHC).

Methods: Formalin-fixed, paraffin-embedded (FFPE) tumor blocks from head and neck squamous cell carcinoma (HNSCC) specimen (n=62) were cut to sections of 3 μm thickness, deparaffinized and rehydrated by incubation in xylene and graded concentrations of ethanol. Antigen retrieval was performed by heat induced epitope retrieval in sodium/citrate buffer (pH 6.0), heated in a microwave at 900 watt for 5 minutes with subsequent heating for another 10 minutes at 450 watt. Endogenous peroxidases were blocked by immersion in 0.3% hydrogen peroxide in methanol for 30 minutes after which samples were blocked with 10% normal goat serum (Dako, X0907) for 30 minutes at room temperature. Slides were incubated for 1 h at room temperature with 5 μg/ml CD142-FITC clone HTF-1 (MACS Miltenyi Biotec, 130-098-853) or isotype control (IgG1-FITC, Biolegend, 400107) in antibody diluent (phosphate buffered saline (PBS)+0.1% and 2% normal goat serum (NGS)), then using 2.5 μg/ml rabbit anti-FITC (Thermo Fisher, 71-1900) in antibody diluent for 30 minutes at room temperature. Staining was visualized with a poly-HRP anti-rabbit/mouse IgG ready to use solution (Immunologic DPVB55HRP, bright vision) for 30 minutes at room temperature and using 3,3′-Diaminobenzidine (DAB, Sigma, D5637). All tissue sections were briefly rinsed in water and counterstained with Mayer's haematoxylin (Merck, 092490500) for 1 minute. Slides were then rinsed again in water and mounted in Kaiser Glycerol gelatin (Merck, 1092420100).

The percentage of tumor cells within the tumor area (with a minimal of 100 tumor cells) of HNSCC patient tumors was estimated by a HNSCC pathologist on haematoxylin and TF-DAB stained slides. Pictures of the immunostained slides were taken using a LEICA 6000 DM microscope (LEICA, SOLMs, Germany) and Leica software. Details regarding the patient samples are summarized below in Table 1.

TABLE 1
Origin of HNSCC patient tumor cells.

| | | N | % | Mean [range] |
|---|---|---|---|---|
| Sex | Male | 36 | 59 | |
| | Female | 25 | 41 | |
| Age at diagnosis | | | | 63 [22-81] |
| Anatomical tumor location | oral cavity | 39 | 64 | |
| | larynx | 3 | 5 | |
| | oropharynx | 19 | 31 | |
| | HPV− | 17 | 90 | |
| | HPV+ | 1 | 5 | |
| | unknown | 1 | 5 | |
| Pathological stage (pT-stage) | T1 | 5 | 8 | |
| | T2 | 25 | 41 | |
| | T3 | 11 | 18 | |
| | T4 | 20 | 33 | |
| TNM-8 classification | I | 3 | 5 | |
| | II | 12 | 20 | |
| | III | 9 | 15 | |
| | IVA | 32 | 52 | |
| | IVB | 5 | 8 | |

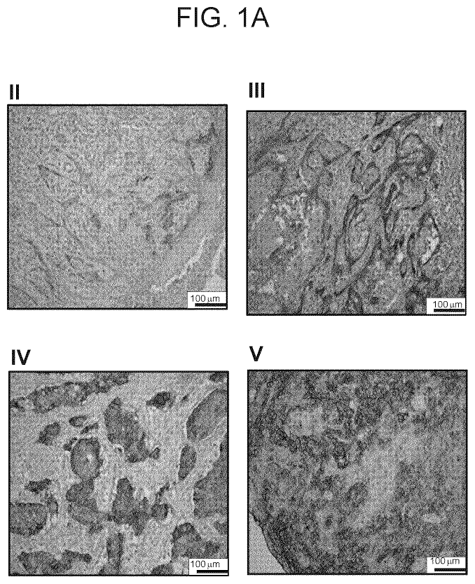

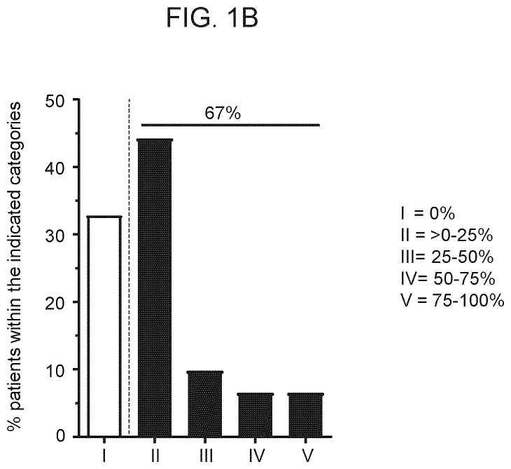

Results: TF expression was observed to be variable between tissue samples. (FIG. 1A). The levels of TF expression could be placed into five different categories (buckets), with I being negative for TF expression and II-V displaying increasing amounts of TF molecules (II: >0-25%, III: >25-50%, IV: >50-75%, V: >75%). Of the samples analyzed, approximately 67% of the tumor specimens showed expression of TF (FIG. 1B). The majority of the TF-positive tumors demonstrated a maximum of 25% positive tumor cells within the tumor area (II). When present, TF was predominantly localized on the membrane, sometimes combined with cytoplasmic localization.

### Example 1B: Tissue Factor (TF) is Expressed in HNSCC Cell Culture Models

HNSCC arises from human papilloma virus (HPV) infection, or through exogenous carcinogen exposure. HNSCC caused by HPV is currently considered a separate disease entity. In this example, experiments were performed to show that HNSCC human mucosal cell lines that were either HPV+ or HPV− had elevated TF expression compared to non-cancerous primary cells.

Methods: Primary human mucosal cells were isolated from excised tumor specimen of patients treated by uvulopalatopharyngeoplasty as described previously and cultured in Dulbecco's Modified Eagle's Medium (DMEM), containing 5% fetal bovine serum (FBS) and 2 mM L-glutamine.

Keratinocytes were cultured in serum-free Keratinocyte medium (KSFM, Gibco, 17005-034) supplemented with 0.1% bovine serum albumin (BSA, Biovision), recombinant epidermal growth factor (EGF) (Gibco, 10450-013) and bovine pituitary extract (Gibco, 13028-014). HNSCC cell lines were cultured in Dulbecco's Modified Eagle's Medium (DMEM), containing 5% fetal bovine serum (FBS, Gibco, 10270-106) and 2 mM L-glutamine (Lonza, BE-17-605E). The FaDu cell line was purchased from the ATCC. The cell lines UM-SCC-14A, UM-SCC-14B, UM-SCC-22A, UM-SCC-22B, UM-SCC-47 (HPV+) and UM-SCC-104 (HPV+) were described previously (Lin et al., “Head and neck squamous cell carcinoma cell lines: established models and rationale for selection,” Head Neck 2007; 29:163-88). The VU-SCC-017, VU-SCC-078, VU-SCC-080, VU-SCC-094, VU-SCC-096, VU-SCC-120, VU-SCC-1365, VU-SCC-OE and VU-SCC-RO lines were also described previously (Hermsen et al., “Centromeric breakage as a major cause of cytogenetic abnormalities in oral squamous cell carcinoma,” Genes Chromosomes Cancer 1996; 15:1-9; and van Zeeburg et al., “Generation and molecular characterization of head and neck squamous cell lines of fanconi anemia patients,” Cancer Res. 2005; 65:1271-6). Cell line details are described in Table 2, below.

TABLE 2
HNSCC cell line characteristics.

| HNSCC cell | | | Primary tumor |
|---|---|---|---|
| line | Sex | Stage | site |
| FaDu | Male | NA | Hypopharynx |
| UM-SCC-14A | Female | T1N0 | Floor of mouth |
| UM-SCC-14B | Female | T1N0 | Floor of mouth |
| UM-SCC-22A | Female | T2N1 | Hypopharynx |
| UM-SCC-22B | Female | T2N1 | Neck metastasis |
| | | | of UM-SCC-22A |
| UM-SCC-47⁺ | Male | T3N1 | Oral cavity |
| UM-SCC-104⁺ | Male | recurrence | Floor of mouth |
| | | (T4N2b) | |
| VU-SCC-017 | Female | T2N2b | Oral cavity |
| VU-SCC-078 | Female | T3N2b | Tongue |
| VU-SCC-080 | Male | recurrence | Base of tongue |
| VU-SCC-094 | Female | T3N1 | Tongue |
| VU-SCC-96 | Male | T4N1 | Retromolar trigone |
| VU-SCC-120 | Female | T3N1 | Tongue |
| VU-SCC-1365^{F} | Male | NA | Mouth mucosa |
| VU-SCC-OE | Male | Lymph node | Floor of mouth |
| | | metastasis | |
| VU-SCC-RO | Male | T3N2b | Oropharynx |

⁺= HPV-positive tumor
^{F}= Fanconi anemia HNSCC

Cell lines were authenticated by microsatellite PCR profiling and TP53 sequencing. HPV status was confirmed by a GP5+/6+ DNA PCR (Smeets et al., “A novel algorithm for reliable detection of human papillomavirus in paraffin embedded head and neck cancer specimen,” Int. J. Cancer 2007; 121:2465-72). All cells were maintained at 37° C. in 5% CO₂.

TF expression was quantified by detecting TF cell surface expression using a mouse anti-human TF antibody (R&D systems, clone 323514, MAB2339), with the use of a mouse isotype murine IgG₂ₐ (BD Pharmingen, 555571) as a negative control. Expression was quantified using QIFIKIT analysis (DAKO K0078) and measured on an LSR-Fortessa™ cell analyzer (BD Biosciences, San Diego, CA) and analyzed with DIVA software version 8.0 or FCS Express 6.0.

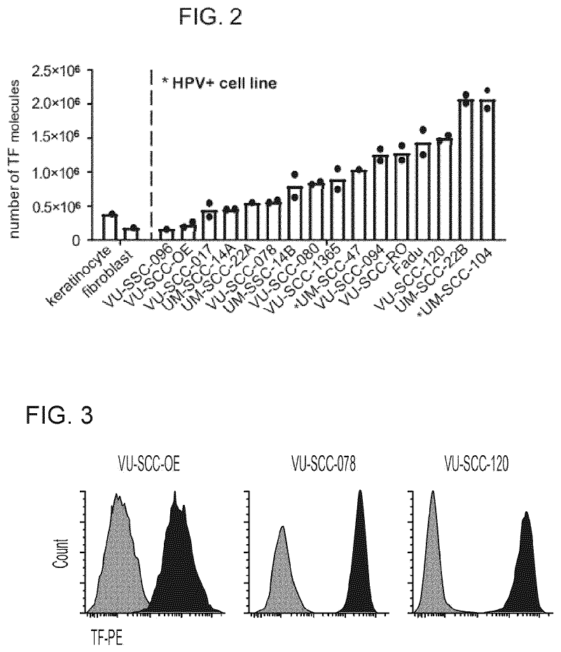

Results: With the exception of VU-SCC-OE and VU-SCC-096, the majority of tested HNSCC cell lines demonstrated higher numbers of TF molecules compared to primary keratinocytes and fibroblasts which served as normal cell references (FIG. 2). TF expression was found to be the highest in UM-SCC-22B and UM-SCC-104 (FIG. 2).

### Example 1C: Tisotumab Binds HNSCC Cells that Express TF

Methods: Three VU-SCC cells lines with various levels of TF expression were selected for additional analysis. It was determined that VU-SCC-OE had TF expression levels similar to that of non-cancerous fibroblasts and VU-SCC-078 had slightly higher expression levels of TF compared to VU-SCC-OE; VU-SCC-120 was a cell line with high levels of TF expression (FIG. 2). These three cell lines were cultured and subsequently assayed for tisotumab binding. Cells were incubated with the anti-human TF monoclonal antibody, (tisotumab, 2 μg/ml, IgG1-1015-011, Genmab) or an isotype control IgG (Genmab IgG1-b12). The PE-conjugated goat (Fab′)2 anti-human IgG secondary antibody (1:50 Jackson, 109-116-098) was applied and the cells were analyzed by flow cytometry.

Results: Binding of tisotumab, the antibody portion of tisotumab vedotin, was validated by flow cytometry on a selection of HNSCC cell lines with varying levels of TF molecules on their cell surface (FIG. 3). Tisotumab bound robustly to HNSCC tumor cell lines VU-SCC-078 (intermediate expression of TF) and VU-SCC-120 (high levels of TF) (FIG. 3). Tisotumab was still capable of binding VU-SCC-OE, which represented HNSCC cells with lower levels of TF expression (FIG. 3). Staining with IgG-isotype control served as background control.

### Example 2: TV Induces Dose-Dependent Tumor Cell Killing in HNSCC Cells

In the preceding Example, it was shown that tisotumab was capable of binding TF-expressing cells derived from HNSCC. The cytotoxic effects of the antibody drug conjugate tisotumab vedotin (TV) on HNSCC cells in culture are show in this Example.

Methods: Cells were seeded in 96-wells plates on day 0 (1000-6000 cells/well, depending on the cell line). After overnight culturing, tisotumab vedotin or the negative control isotype IgG₁-vedotin (IgG₁-c-b12-vc monomethyl auristatin E (MMAE)) was added in increasing concentrations to the different wells. The relative number of viable cells was assessed on day 5 using a Cell Titer-Blue assay (Promega, Leiden, The Netherlands, G8080) according to the protocol from the manufacturer and fluorescence was measured using the GloMax®-Multi Detection System (Promega). Staurosporine (5 μM, Sigma, UCN-01 or 56942) was used as a positive control to induce maximum cell death, and untreated cells were also used as a negative control. After subtraction of background (medium only), the percentages of viable cells were calculated using the following formula: % viable cells=((mean fluorescence test samples−mean fluorescence staurosporine sample)/(mean fluorescence untreated sample−mean fluorescence staurosporine sample))*100. Graphs represent non-linear regression curves of log-transformed antibody concentrations, using Graphpad Prism version 9.1.0 (GraphPad Software, San Diego, CA).

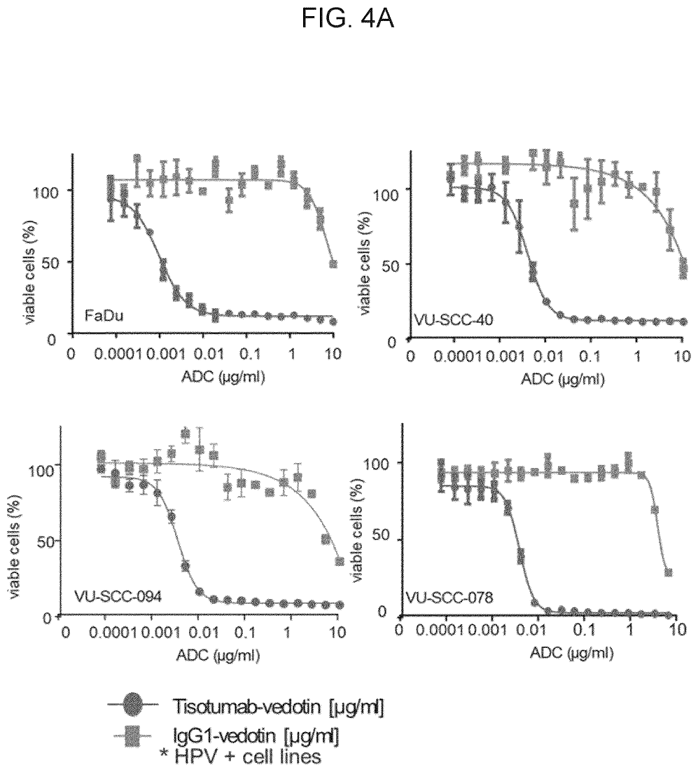

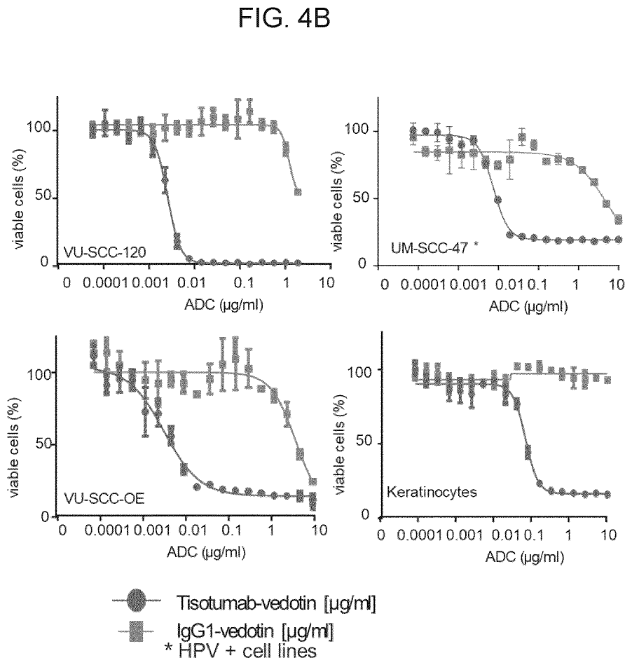

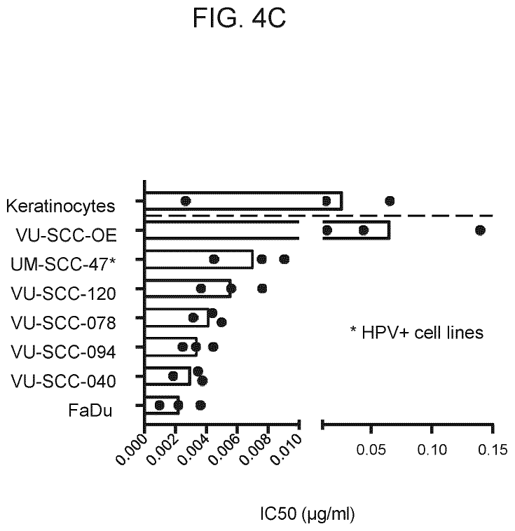

Results: Tisotumab vedotin (TV) showed efficient and dose-dependent killing of HNSCC cell lines (FIGS. 4A-4B). Effective cell killing was observed at low concentrations of TV (FIGS. 4A-C). Cells with low TF expression such as primary keratinocytes and the HNSCC cell line VU-SCC-OE demonstrated an average IC50 dose of above 0.025 μg/ml, in contrast to mean IC50 values ranging from 0.0025-0.007 μg/ml for HNSCC cell lines with intermediate and high levels of TF expression (FIGS. 4A-4C). The non-targeted isotype IgG1-vedotin control antibody did not demonstrate cell killing in HNSCC cell lines until 100-fold increased dose was used compared to TV (FIGS. 4A-4B). This indicated that binding of tisotumab vedotin to TF was essential for efficient killing of the HNSCC tumor cells.

### Example 3a: HNSCC Cells Injected into Mice Result in TF-Expressing Solid Tumors

This Example demonstrated that the various HNSCC cell lines could be used to establish xenografts with differential TF expression profiles.

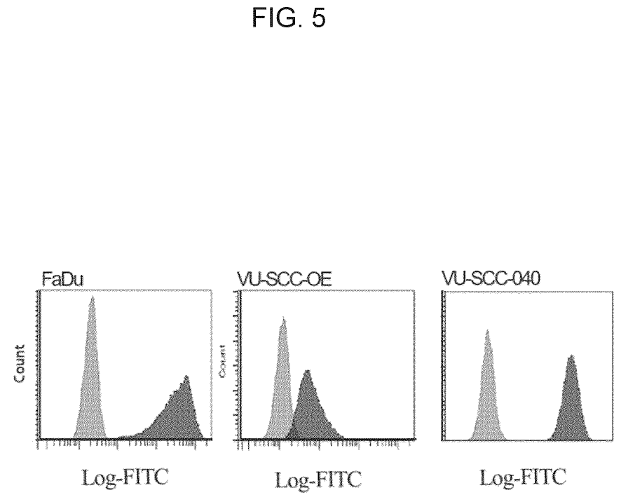

Methods: Three HNSCC cell lines that were used to induce human xenograft tumors in mice were assessed for TF expression by flow cytometry. Cells were stained with either 7.5 μg/ml human anti-TF antibody (CD142-FITC, Macs Miltenyi Biotec, 130-098-853) or the isotype control mouse IgG1-FITC (Biolegend, 400170). The three selected cell lines were all shown to express TF (FIG. 5).

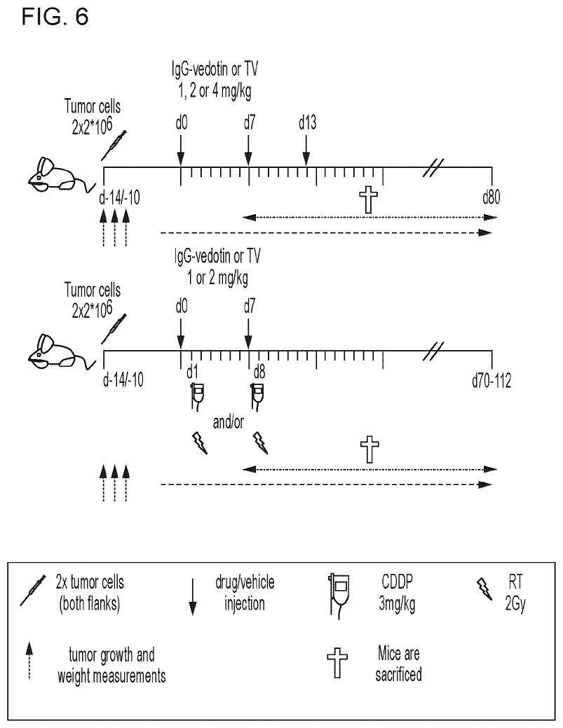

Nude mice (female, athymic nu/nu, age 6-8 weeks old, from Envigo, Horst, The Netherlands), were kept in filter top cages under sterile conditions under standardized environmental conditions. The HNSCC cell lines FaDu, VU-SCC-OE or VU-SCC-040 were subcutaneously injected in both flanks (2×10⁶ cells per flank). The implanted tumor cells were allowed to grow to an average size of 100 mm³ (range 40-180 mm³). The mice were treated intraperitoneally (ip) with tisotumab vedotin or an isotype control (IgG1-b12-c-vcMMAE) (FIG. 6). Mice were sacrificed when the tumor volumes reached 5-times the start tumor volume in one of the 2 flanks and/or displayed tumor ulceration, or the mice displayed a body weight loss≥20% or a moribund appearance. Tumor volume was measured with electronic calipers (V=(L×W×H)×0.5 where V=volume, L=length, W=width, H=height) and calculated as mean of tumor(s) per mouse. Tumors with a start volume below 40 mm³ and no signs of growth were excluded from the analysis. Tumors from un-treated tumor bearing mice were harvested and embedded in paraffin for analysis of TF expression using IHC analysis as described in the preceding examples. TF expression was detected using anti-CD142-FITC or isotype control (IgG1-FITC) with rabbit anti-FITC (Zymed) and BrightVision Immunohistochemistry (IHC) Detection Kit and visualized with DAB counterstained with Haematoxylin.

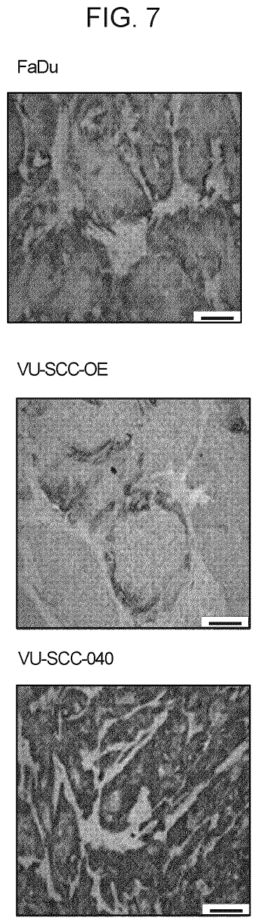

Results: Tumors that were isolated from xenograft mice all demonstrated TF expression (FIG. 7). FaDu and VU-SCC-040 demonstrated abundant TF expression in culture, which corresponded to the high expression of TF found within the in vivo grown tumors. FaDu and VU-SCC-040 xenografts exhibited TF expression V>75% (FIG. 7). The VU-SCC-OE cell line, which consistently had lower TF expression (FIGS. 2 and 5) also demonstrated fewer TF positive tumor cells within the xenograft tumor [TF expression bucket II>0-25%](FIG. 7).

### Example 3B: TV Monotherapy Decreases Tumor Volume in Xenograft Mice by Day 7

This Example demonstrates that tisotumab vedotin (TV) monotherapy inhibited tumor growth in xenograft mice.

Nude mice were injected with HNSCC cells and xenografts were established as described above in Example 3A. Xenograft mice (5 mice with 8-10 tumors per group) were treated with PBS, 2 and 4 mg/kg tisotumab vedotin, or 4 mg/kg isotype IgG1-vedotin on days 0, 7 and 13 (FaDu) or on days 0, 7 and 14 (VU-SCC-OE and VU-SCC-040) (FIG. 6). Tumor volume was measured as described in the preceding example. Data analysis was performed using Graph Pad Prism version 9.1.0 (GraphPad Software, San Diego, CA). Data are depicted as mean±SD or mean±SEM. Statistical differences were determined using a Kruskal-Wallis test with multiple comparison tests (>2 groups). Improved overall survival was assessed using the Kaplan-Meier method and differences between the groups was assessed by the Log-rank test (Mantel-Cox). p-values<0.05 were considered significant (*p<0.05, **p<0.01,***p<0.001, **** p<0.0001, ns=not significant).

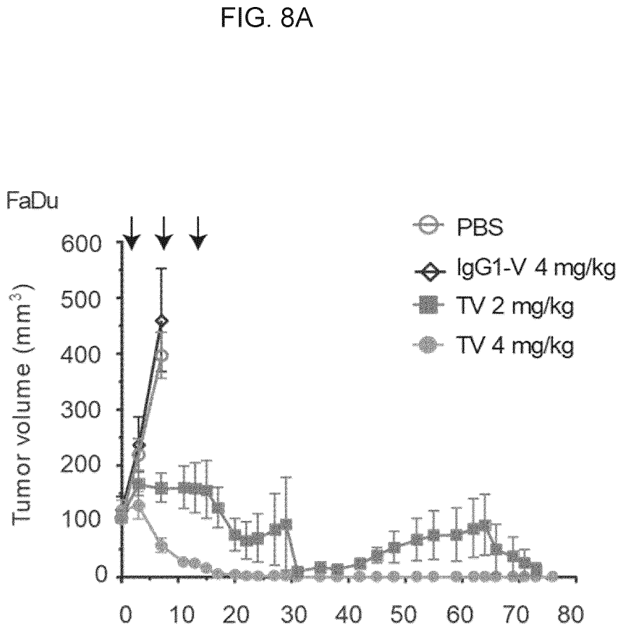

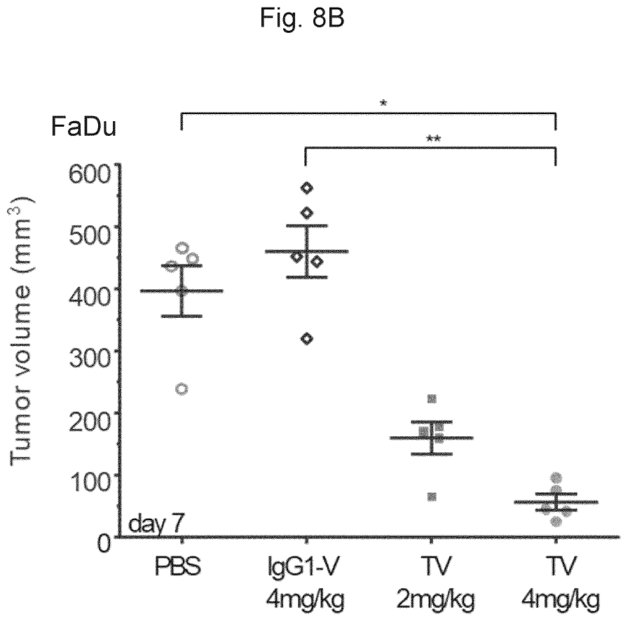

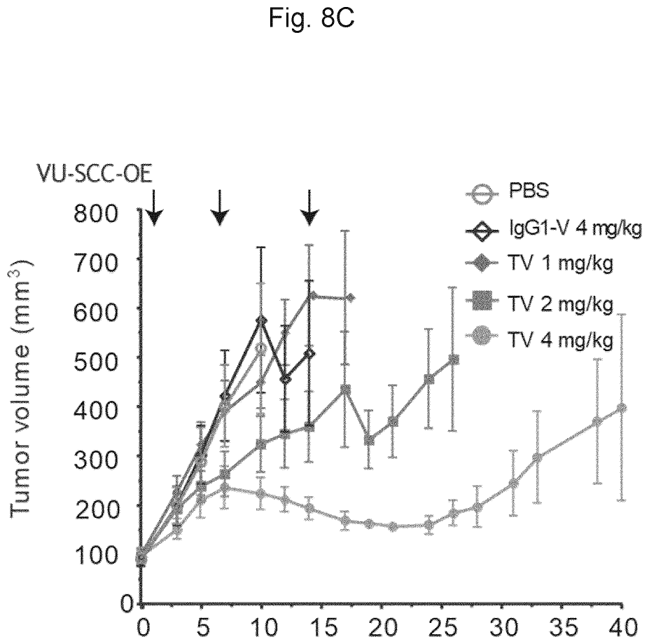

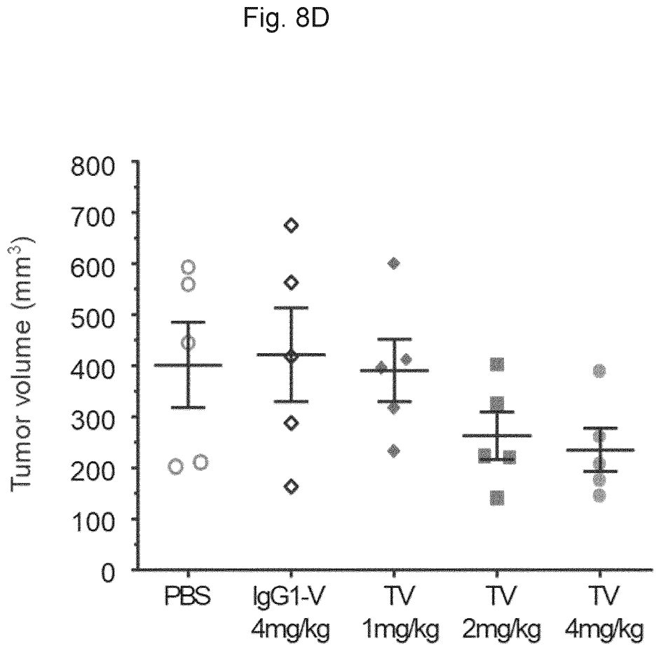

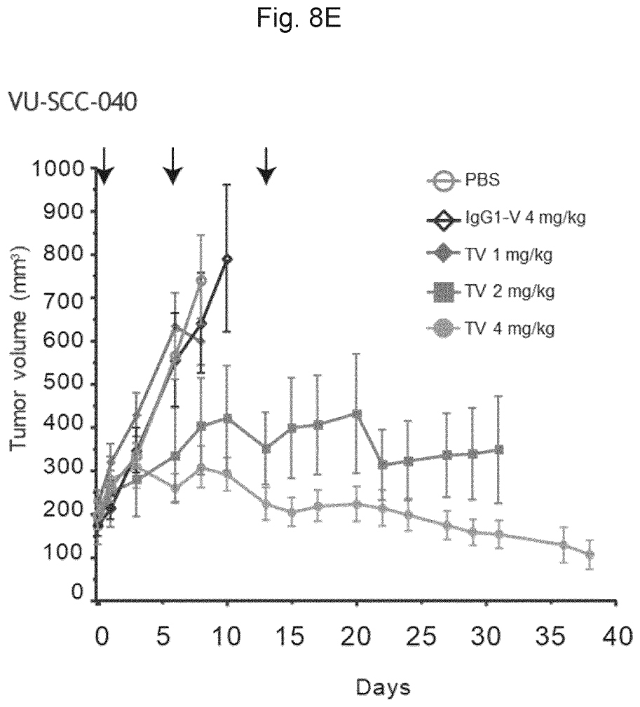

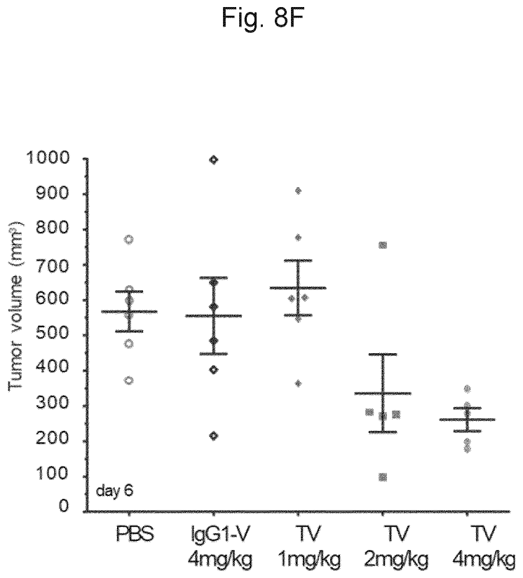

Results: FaDu mice treated with either PBS or IgG1-vedotin demonstrated rapid tumor growth and most mice had to be sacrificed at day 7 (FIGS. 8A and 8B). Mice treated with 2 mg/kg TV demonstrated significant inhibition of tumor growth compared to the mice from the control groups and tumor regression was observed after 3 treatments (FIG. 8A). After treatment discontinuation (last treatment on day 13), tumors started to regrow, which was visible from 30 days after start of the initial treatment. Mice treated with 4 mg/kg TV demonstrated significant and persistent tumor regression after the first treatment (FIG. 8A). Moreover, complete tumor regression was observed in this group within 30 days in all mice without recurrence of tumor growth until the end of the experiment (day 76) (FIG. 8A). Comparison of tumor volumes between treatment groups on day 7, the last day that all treatment groups were complete, confirmed considerably lower tumor size in mice that been treated with tisotumab vedotin compared to the isotype control or PBS (FIG. 8B).

Treatment of mice bearing tumors (day 0, 7 and 14) induced by injection with the low TF-expressing VU-SCC-OE cell line demonstrated rapid tumor growth in both the control groups (mice treated with PBS or IgG₁-V) but also when mice were treated with low dose of TV (1 mg/kg) (FIGS. 8C and 8D). Higher treatment doses of 2 and 4 mg/kg resulted in inhibition of tumor growth.

VU-SCC-040 tumor bearing mice treated with 4 mg/kg TV demonstrated, after 1 treatment, reduced tumor growth at day 7 compared to mice treated with the isotype control (FIGS. 8E and 8F). 1 mg/kg TV monotherapy did not inhibit tumor growth compared to the control groups, but inhibition of tumor growth was observed with the 2 mg/kg and in particular with 4 mg/kg TV dose (FIGS. 8E and 8F).

### Example 4: TV Improves the Anti-Tumor Activity of Chemoradiotherapy (CRT) In Vivo in HNSCC Xenograft Models

Concurrent chemoradiotherapy (CRT) is the current standard of care for patients with locally advanced HNSCC outside the oral cavity. However, 30-40% of patients experience tumor recurrence despite this highly invasive treatment. In this Example, TV was assessed for the ability to preclinically inhibit tumor growth and prolong survival when combined with standard chemoradiotherapy in HNSCC xenograft models.

Methods: FaDu xenografts were established in nude mice (5-6 mice/group) as described above in Example 3. In combination therapies, the modification was made so that mice were sacrificed when the tumor volume reached 10× the start tumor volume in one of the 2 flanks and/or displayed tumor ulceration, or when the mice displayed a body weight loss≥20% or showed a moribund appearance.

FaDu xenograft mice were treated 24 hour hours prior to CRT (CRT was administered on day 1 and 11) with TV or the isotype control, which was administered on day 0 and 10. VU-SCC-OE xenograft mice were treated 24 hour hours prior to CRT (CRT was administered on day 1 and 8) with TV or the isotype (IgG1-vedotin) control, which was administered on day 0 and 7. For mice receiving CRT, whole body irradiation (2 Gy; Varian TrueBeam™ Linear Accelerator) was applied approximately 4 hours after administering cisplatin (CDDP, 3 mg/kg). Tumor volume was measured as described above, and statistical analysis was performed on the collected data as also described above.

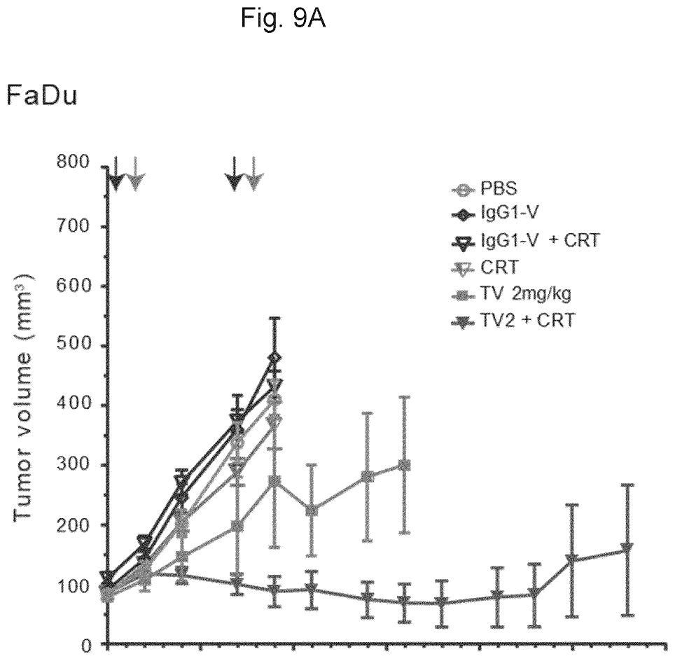

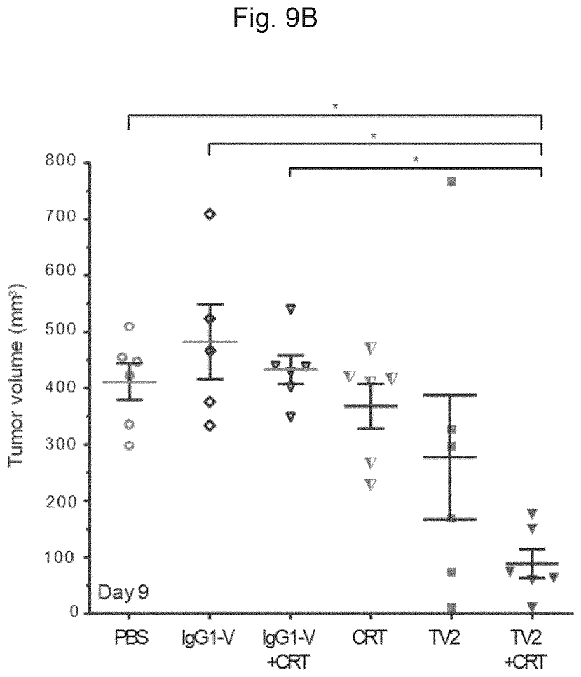

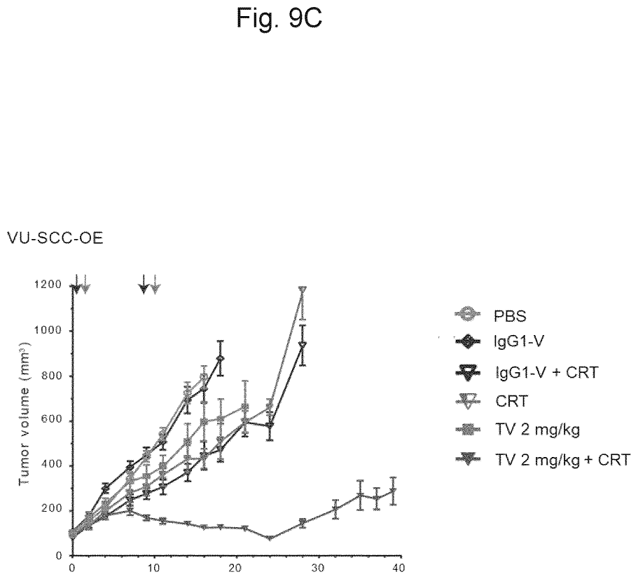

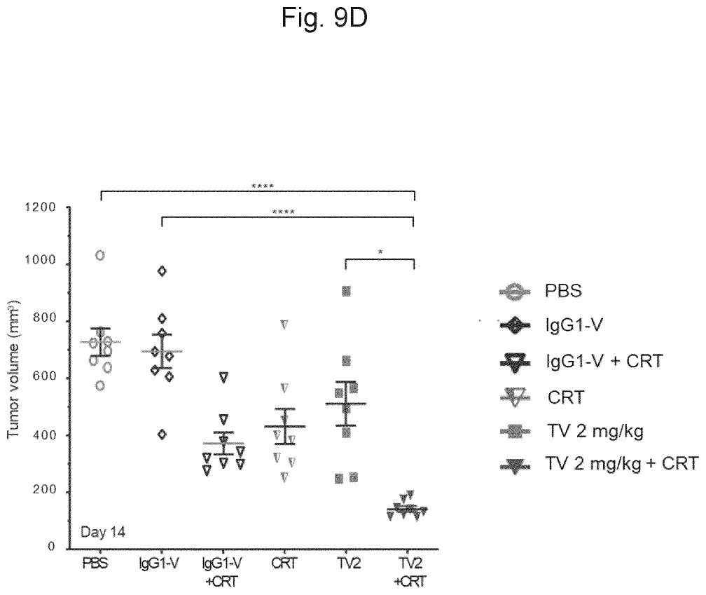

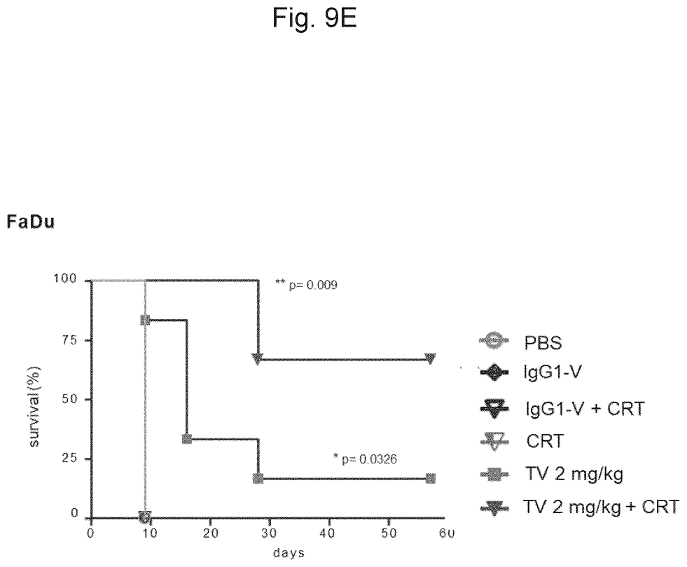

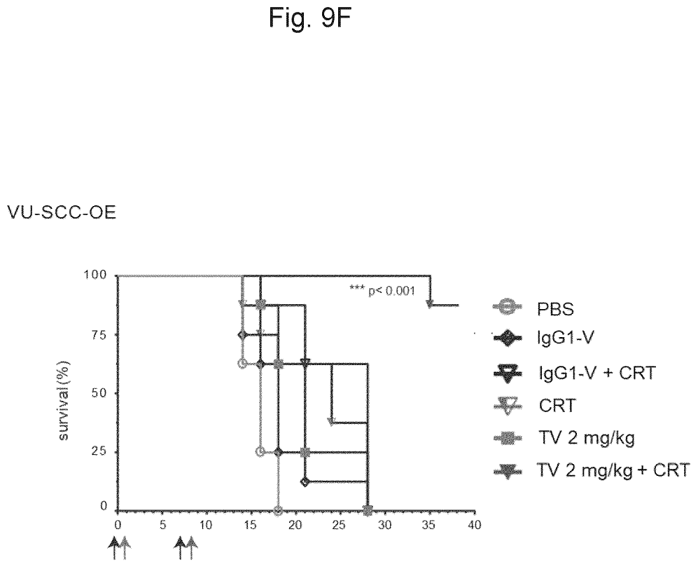

Results: FaDu xenograft mice from the negative control groups (treated with PBS or IgG1-vedotin (IgG1-V), demonstrated rapid tumor growth, and treatment with CRT or IgG1-V+CRT) did not have a significant impact on tumor growth (FIGS. 9A and 9B). In contrast, the mice treated with the dose of 2 mg/kg TV showed reduced tumor growth compared to the IgG1-vedotin control group, though variation within the treated animals was observed (FIG. 9A). Although CRT itself did not inhibit tumor growth in this model, when combined with TV, a significant reduction in tumor size was observed at day 9 after first treatment compared to the control group receiving IgG1-vedotin combined with CRT (FIGS. 9A and 9B).

In the low TF-expressing VU-SCC-OE xenograft tumor model, there were no differences in tumor growth between the PBS treated mice and mice treated with IgG₁-vedotin. Treatment with CRT (+/−IgG1-V) or 2 mg/kg TV showed some inhibition of tumor growth compared to the control treatment groups (PBS and IgG₁-vedotin). In contrast, the combination of CRT with 2 mg/kg of TV (FIGS. 9C and 9D) induced tumor regression.

In FaDu xenograft mice, the combination of CRT and TV resulted in a significant increase in overall survival of mice compared to controls groups, or CRT or TV alone as demonstrated by Kaplan-Meir analysis (FIG. 9E). Also in the low TF expressing VU-SCC-OE xenograft tumor model, combination treatment resulted in significantly prolonged survival compared to control groups or CRT or TV alone (FIG. 9F).

These data show that treatment with TV synergized with standard chemoradiotherapy in xenograft HNSCC models, significantly inhibiting tumor growth and prolonging survival of tumor-bearing mice compared to chemoradiotherapy or tisotumab vedotin alone.

### Example 5a: Xenograft Mice have Reduced Tumor Sizes and Enhanced Survival Outcomes when Treated with Tisotumab Vedotin in Combination with Radiotherapy Compared to Radiotherapy Alone

In this Example, TV was assessed for the ability to preclinically inhibit tumor growth and prolong survival when combined with radiotherapy in HNSCC xenograft models.

Methods: The combined effect of tisotumab vedotin (TV) and radiotherapy (RT) was assessed by administering TV doses at 1 mg/kg and 2 mg/kg as monotherapy or in combination with RT compared with RT alone. FaDu xenografts were established in nude mice as described in Example 3. Tisotumab vedotin (TV) or control IgG1-vedotin (IgG1-V) was administered at day 0. For mice subjected to RT, 2 Gy whole body radiotherapy was administered at day 1. A minimum of 8 tumors were included per group.

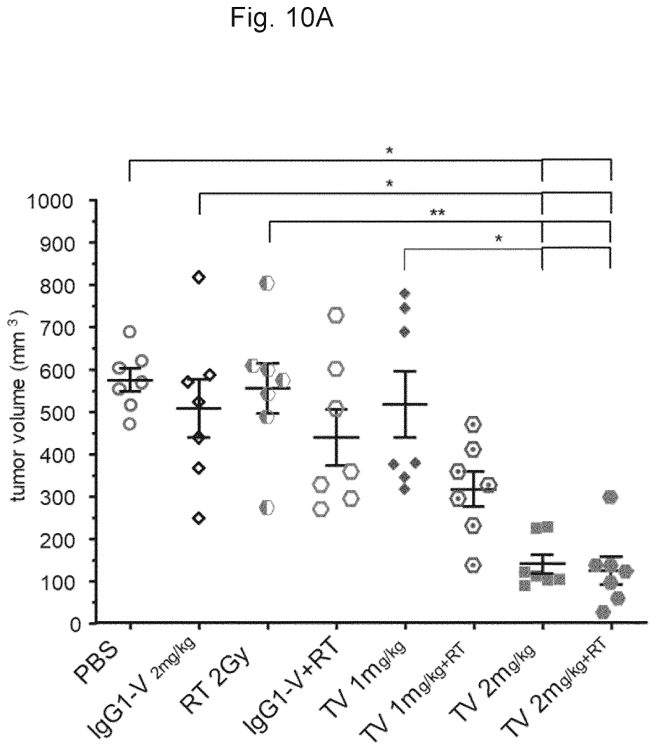

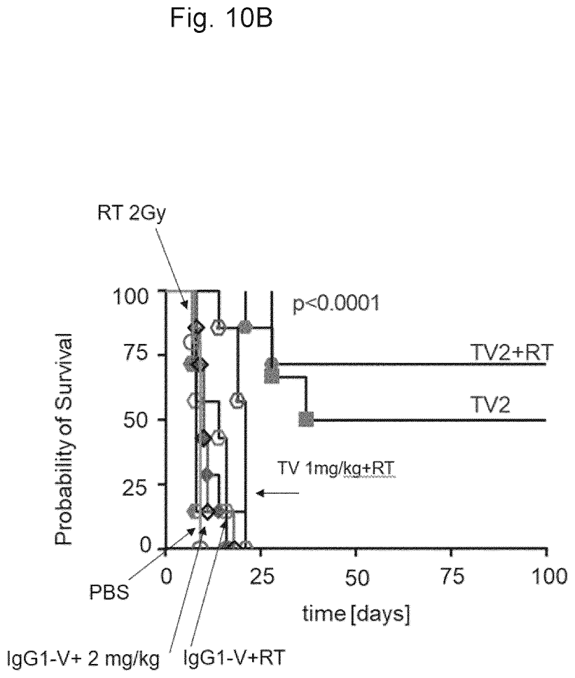

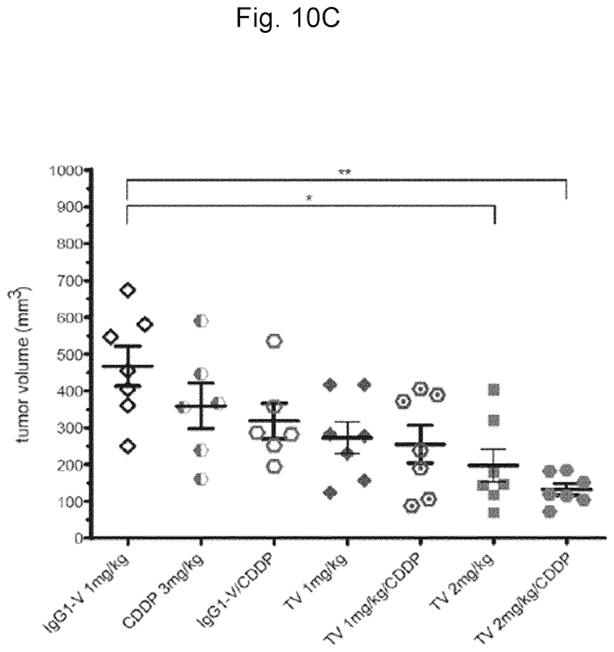

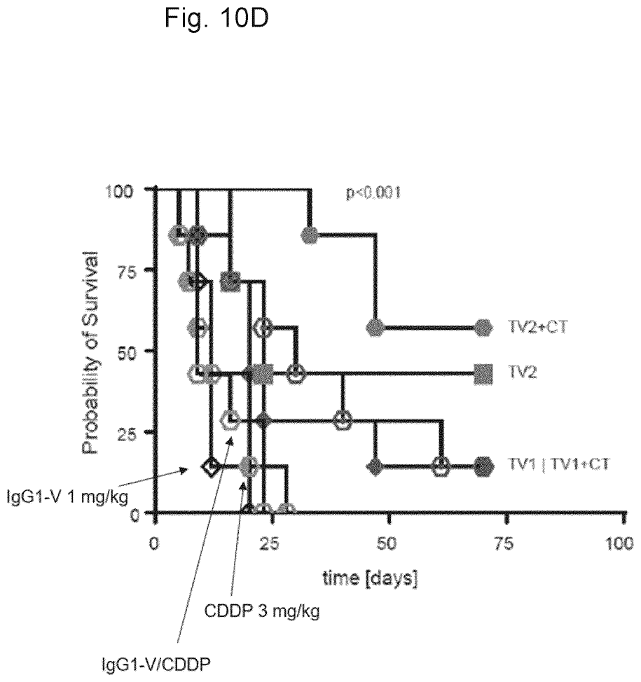

Results: Mice treated with TV 2 mg/kg and TV 2 mg/kg+RT had the lowest tumor burdens on day 7 (FIG. 10A) as compared to RT alone or with TV administered at 1 mg/kg (alone or while used in combination with RT).

Treatment with RT alone, IgG-V+RT or 1 mg/kg TV alone did not prolong survival of tumor-bearing mice compared to control treatment (PBS or IgG-V). At day 7, none of the IgG1-vedotin control treatments decreased the tumor volume of the mice. Decreased tumor volume was observed with 1 mg/kg TV and 2 mg/kg TV when combined with radiotherapy (FIG. 10A). Notably, significantly better survival time was observed in the animals treated with 2 mg/kg TV+RT compared to the animals treated with RT alone (p<0.001) (FIG. 10B). The medians and ranges of the survival rates of the therapies were reported in Table 3 below. Mice that received 2 mg/kg TV+RT were more likely to survive compared to 2 mg/kg TV alone. The median survival for 2 mg/kg TV+RT combination therapy could not be calculated because the median was not reached by the end of the experiment (day 112).

Though the median survival did differ between 2 mg/kg TV monotherapy (69.5 days) and 2 mg/kg TV+RT (median not reached at the end of the experiment, day 112), there was no significant difference in survival between the groups (FIG. 10B, Table 3, below). The data from these in vivo experiments suggest that tisotumab vedotin can improve the clinical efficacy of RT in the treatment of HNSCC.

TABLE 3
Median Survival of Combined Treatment TV and Radiotherapy

| | | hIgG-V | hTV1 | hTV | RT | hIgG-V + | hTV1 + | hTV2 + |
|---|---|---|---|---|---|---|---|---|
| | PBS | 2 mg/kg | mg/kg | 2 mg/kg | 2 Gy | RT | RT | RT |
| Median | 9 | 10 | 10 | 69.5 | 8 | 14 | 21 | Undefined |
| survival | | | | | | | | |
| [days] | | | | | | | | |
| Range | 7-10 | 8-18 | 8-16 | 28-112* | 7-16 | 8-21 | 14-21 | 21-112* |
| Survival | | | | | | | | |
| [days] | | | | | | | | |

*End of the experiment is day 112

### Example 5B: Xenograft Mice have Reduced Tumor Sizes and Enhanced Survival Outcomes when Treated with Tisotumab Vedotin in Combination with Chemotherapy Compared to Chemotherapy Alone

In this Example, TV was assessed for the ability to preclinically inhibit tumor growth and prolong survival when combined with chemotherapy in HNSCC xenograft models.

Methods: The combined effect of tisotumab vedotin (TV) and cisplatin therapy (CDDP, a form of platinum-based chemotherapy) was assessed by administering TV doses at 1 mg/kg and 2 mg/kg as monotherapy, or combined with cisplatin therapy, as opposed to cisplatin therapy alone. FaDu xenografts were established in nude mice (6-7 mice/group) as described above in Example 4. Mice were treated with the antibodies on day 0 and 7 and/or on day 1 and 8 with 3 mg/kg intraperitoneal cisplatin (CDDP) chemotherapy (CT) (FIG. 6). TV or control IgG-vedotin (IgG1-V) was administered at day 0. For mice subjected to chemotherapy, cisplatin was administered at day 1. A minimum of 8 tumors were included per group.

Results: Additionally, the combined effect of TV to cisplatin treatment was assessed and it was observed that addition of 2 mg/kg TV to cisplatin therapy significantly reduced tumor load on day 7 and improved survival compared to either treatment alone (FIGS. 10C and 10D)

What is claimed is:

1. A method of treating cancer in a subject, the method comprising:
(i) administering to the subject a radiation therapy; and
(ii) administering to the subject an antibody-drug conjugate that binds to tissue factor (TF), wherein the antibody-drug conjugate comprises an anti-TF antibody or an antigen-binding fragment thereof conjugated to an auristatin or a functional analog thereof or a functional derivative thereof.

2. The method of claim 1, wherein the auristatin is monomethyl auristatin or a functional analog thereof or a function derivative thereof.

3. The method of claim 1 or claim 2, wherein the auristatin is monomethyl auristatin E (MMAE).

4. The method of any one of claims 1-3, wherein the antibody-drug conjugate is administered at a dose ranging from about 0.9 mg/kg to about 2.1 mg/kg.

5. The method of any one of claims 1-4, wherein the antibody-drug conjugate is administered at a dose ranging from about 0.9 mg/kg to about 1.7 mg/kg.

6. The method of any one of claims 1-5, wherein the antibody-drug conjugate is administered at a dose of about 1.3 mg/kg.

7. The method of any one of claims 1-5, wherein the antibody-drug conjugate is administered at a dose of about 1.7 mg/kg.

8. The method of any one of claims 1 to 4, wherein the antibody-drug conjugate is administered at a dose of about 2.0 mg/kg.

9. The method of any one of claims 1-8, wherein the antibody-drug conjugate is administered once about every 1 week, once about every 2 weeks, once about every 3 weeks or once about every 4 weeks.

10. The method of any one of claims 1-9, wherein the antibody-drug conjugate is administered once about every 2 weeks.

11. The method of any one of claims 1-9, wherein the antibody-drug conjugate is administered once about every 3 weeks.

12. The method of any one of claims 1-11, wherein the radiation therapy is at a dose between about 1 Gy and about 100 Gy, such as at a dose of between about 10 Gy and about 70 Gy, such as such as at a dose of between about 30 Gy and about 60 Gy, such as at a dose of between about 40 Gy and about 50 Gy.

13. The method of any one of claims 1-12, wherein the radiation therapy is selected from the group consisting of intensity-modulated radiation therapy (IMRT), image-guided radiation therapy (IGRT), tomotherapy, stereotactic radiosurgery, stereotactic body radiation therapy, photon beam, electron beam, and proton therapy.

14. The method of any one of claims 1-13, wherein the method further comprises administering to the subject a chemotherapeutic agent.

15. The method of claim 14, wherein the chemotherapeutic agent is a platinum-based agent.

16. The method of claim 15, wherein the platinum-based agent is administered at a dose between about AUC=4 and about AUC=6.

17. The method of claim 15 or claim 16, wherein the platinum-based agent is administered a dose of about AUC=5.

18. The method of any one of claims 15-17, wherein the platinum-based agent is administered once about every 1 week, once about every 2 weeks, once about every 3 weeks or once about every 4 weeks.

19. The method of any one of claims 15-18, wherein the platinum-based agent is administered once about every 3 weeks.

20. The method of any one of claims 15-18, wherein the platinum-based agent is administered once about every 4 weeks.

21. The method of any one of claims 1-20, wherein the cancer is a solid tumor.

22. The method of any one of claims 1-21, wherein the cancer is a head and neck squamous cell carcinoma.

23. The method of any one of claims 1-21, wherein the cancer is a gynecological cancer.

24. The method of any one of claims 1-21, wherein the cancer is selected from the list consisting of ovarian cancer, endometrial cancer, cervical cancer, perineal tissue cancer, fallopian tube cancer, uterine cancer, vaginal cancer, vulvar cancer, and gestational trophoblastic disease cancer.

25. The method of any one of claims 1-24, wherein the cancer is associated with a primary tumor positive for tissue factor.

26. The method of any one of claims 1-25, wherein the cancer is an early stage cancer.

27. The method of claim 26, wherein the cancer is a stage I or stage II cancer.

28. The method of claim 26 or claim 27, wherein the cancer is not a recurrent cancer.

29. The method of any one of claims 26-28, wherein the cancer is not locally advanced.

30. The method of any one of claims 26-29, wherein the cancer is not metastatic.

31. The method of any one of claims 26-28, wherein the cancer is locally advanced.

32. The method of any one of claims 1-31, wherein the method of treating is a neoadjuvant treatment for the cancer.

33. The method of claim 32, wherein the antibody-drug conjugate and radiation therapy are administered before surgical intervention for the cancer.

34. The method of claim 32 or claim 33, wherein further the platinum-based agent is administered before surgical intervention for the cancer.

35. The method of claim 33 or claim 34, wherein the antibody-drug conjugate and radiation therapy are administered before surgical removal of one or more tumors associated with the cancer.

36. The method of any one of claims 33-35, wherein further the platinum-based agent is administered before surgical removal of one or more tumors associated with the cancer.

37. The method of any one of claims 1-36, wherein the subject has not received prior therapy for the cancer.

38. The method of any one of claims 1-31, wherein the method of treating is an adjuvant therapy for the cancer.

39. The method of claim 38, wherein the antibody-drug conjugate and the radiation therapy are administered after surgical intervention for the cancer.

40. The method of claim 38 or claim 39, wherein further the platinum-based agent is administered after surgical intervention for the cancer.

41. The method of any one of claims 38-40, wherein the antibody-drug conjugate and radiation therapy are administered after surgical removal of one or more tumors associated with the cancer.

42. The method of any one of claims 38-41, wherein further the platinum-based agent is administered after surgical removal of one or more tumors associated with the cancer.

43. The method of any one of claims 1-42, wherein the anti-TF antibody or antigen-binding fragment thereof of the antibody-drug conjugate is a monoclonal antibody or a monoclonal antigen-binding fragment thereof.

44. The method of any one of claims 1-43, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO:2; and
(iii) a CDR-H3 comprising the amino acid sequence of SEQ ID NO: 3; and
wherein the light chain variable region comprises:
(i) a CDR-L1 comprising the amino acid sequence of SEQ ID NO: 4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof are defined by the IMGT numbering scheme.

45. The method of any one of claims 1-44, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

46. The method of any one of claims 1-45, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO: 8.

47. The method of any one of claims 1-46, wherein the anti-TF antibody of the antibody-drug conjugate is tisotumab.

48. The method of any one of claims 1-47, wherein the antibody-drug conjugate further comprises a linker between the anti-TF antibody or antigen-binding fragment thereof and the auristatin.

49. The method of claim 48, wherein the linker is a cleavable peptide linker.

50. The method of claim 49, wherein the cleavable peptide linker has a formula: -MC-vc-PAB-, wherein:
a) MC is: 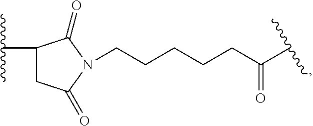 [SMILES: *C(=O)CCCCCN1C(=O)CC(*)C1=O]
b) vc is the dipeptide valine-citrulline, and
c) PAB is:

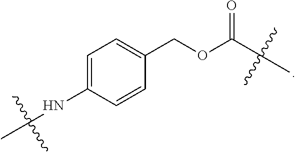 [SMILES: *Nc1ccc(COC(*)=O)cc1]

51. The method of any one of claims 48-50, wherein the linker is attached to sulphydryl residues of the anti-TF antibody obtained by partial reduction or full reduction of the anti-TF antibody or antigen-binding fragment thereof.

52. The method of claim 51, wherein the linker is attached to MMAE, wherein the antibody-drug conjugate has the following structure: 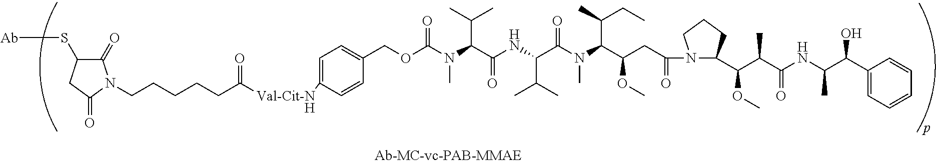
wherein p denotes a number from 1 to 8, S represents a sulphydryl residue of the anti-TF antibody, and Ab designates the anti-TF antibody or antigen-binding fragment thereof.

53. The method of claim 52, wherein the average value of p in a population of the antibody-drug conjugates is about 4.

54. The method of any one of claims 1-53, wherein the antibody-drug conjugate is tisotumab vedotin.

55. The method of any one of claims 1-54, wherein the route of administration for the antibody-drug conjugate is intravenous.

56. The method of any one of claims 15-55, wherein the platinum-based agent is selected from the group consisting of carboplatin, cisplatin, oxaliplatin, and nedaplatin.

57. The method of any one of claims 15-56, wherein the platinum-based agent is carboplatin.

58. The method of any one of claims 15-56, wherein the platinum-based agent is cisplatin.

59. The method of any one of claims 15-58, wherein the route of administration for the platinum-based agent is intravenous.

60. The method of any one of claims 15-59, wherein the platinum-based agent and the antibody-drug conjugate are administered sequentially.

61. The method of any one of claims 15-59, wherein the platinum-based agent and the antibody-drug conjugate are administered simultaneously.

62. The method of any one of claims 1-61, wherein the subject is a human.

63. The method of any one of claims 1-62, wherein the antibody-drug conjugate is in a pharmaceutical composition comprising the antibody-drug conjugate and a pharmaceutically acceptable carrier.

64. The method of any one of claims 15-63, wherein the platinum-based agent is in a pharmaceutical composition comprising the platinum-based agent and a pharmaceutical acceptable carrier.
